(19) United States
(12) Patent Application Publication
(10) Pub. No.: US 2008/0009418 A1
(43) Pub. Date: Jan. 10, 2008

(54) Selective Inhibitors

(75) Inventors: Gerald B. Tometzki, Manly West (AU); Wim Meutermans, Toowong (AU); Bernd Becker, New Farm (AU); Johannes Zuegg, Wynnum (AU); Rajaratnam Premraj, Eight Mile Plains (AU); Craig Muldoon, Eight Mile Plains (AU); Declan McKeveney, Coorparoo (AU); Glenn Condie, Coorparoo (AU)

Correspondence Address: MORRISON & FOERSTER LLP, 755 PAGE MILL RD, PALO ALTO, CA, 94304-1018, US

(73) Assignee: Alchemia LTD., Eight Mile Plains (AU)

(21) Appl. No.: 11/664,632

(22) PCT Filed: Oct. 4, 2005
(86) PCT No.: PCT/AU05/01510

§ 371 Date: Apr. 3, 2007

(30) Foreign Application Priority Data
Oct. 4, 2004 (AU) 2004905675

(51) Int. Cl. C40B 20/00; C07H 15/06; C40B 40/04

(52) U.S. Cl. 506/2; 506/19; 536/17.5

(57) ABSTRACT

A method of identifying biologically active compounds with defined selectivity profile comprises: (c) designing a library of compounds of formula (1) to scan molecular diversity; and (d) assaying the library of compounds in at least two different biological assays.

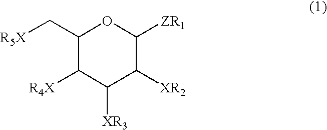

### FIELD OF THE INVENTION

The invention relates to a method of identifying compounds with selective biologically activities, and libraries of compounds.

### BACKGROUND

Small molecules involved in molecular interactions with a biological target, be it enzyme or receptor, are often described in terms of binding elements or pharmacophore groups which directly interact with the target, and non-binding components which form the framework of the bioactive molecule. In the case of peptide ligands or substrates for instance, a number of amino acid side chains usually form direct interactions with their receptor or enzyme, whereas specific folds of the peptide backbone (and other amino acid residues) provide the structure or scaffold that controls the relative positioning of these side chains. In a peptidomimetic approach to drug discovery, the side chains of important amino acids may be systematically modulated to identify better binding interactions. This is referred to as a scanning approach. Unfortunately, the side chains of peptides are rarely independent, such that each interaction cannot be optimised without consideration of the others.

One way to overcome this problem is to construct diversity libraries.

So far, approaches for creating universal diversity have largely focused on the combination of substituents aspects. When it comes to creating diversity in presentation of these substituents, pharmaceutical companies generally turn to the known heterocyclic scaffolds, with an emphasis on the so-called ‘privileged structures’. Creating structural diversity in libraries has been highly desired but has been limited by the lack of structural diversity in the chemically useful scaffolds.

Monosaccharides provide an excellent sugar scaffold to design molecular diversity by appending desired substituents at selected positions around the sugar scaffold. The monosaccharide-based scaffold contains five chiral, functionalized positions, enabling attachment of various substituents at each position. This provides a unique opportunity to create libraries of structurally diverse molecules, by varying the pharmacophoric groups, the scaffold and the positions of attachment of the pharmacophoric groups in a systematic manner. A pharmacophoric group in the context of these libraries is an appended group or substituent, or part thereof, which imparts pharmacological activity to the molecule.

Molecular diversity could be considered as consisting of diversity in pharmacophoric group combinations (diversity in substituents) and diversity in the way these pharmacophoric groups are presented (diversity in shape). Libraries of compounds in which either diversity of substituents, or diversity of shape, or both of these parameters are varied systematically are said to scan molecular diversity.

There is a need for methods to improve the development of drug candidates that purposely interact with selected targets, and not with other targets, in order to minimize side effects. Selectivity profiles are determined by biological assays, either in vitro or in vivo, in which compounds exhibit a specific response in each assay. The panel of specific responses represents the selectivity profile across the selected assays. The profile distinguishes actives against non-actives in each assay. Methods to improve the identification of selectivity profiles overcome or at least partially ameliorate this problem.

In previous applications (WO2004014929 and WO2003082846) we demonstrated that arrays of novel compounds could be synthesized in a combinatorial manner. The libraries of molecules described in these inventions were synthesized in a manner such that the position, orientation and chemical characteristics of pharmacophoric groups around a range of chemical scaffolds, could be modified and/or controlled.

In a later application (WO2004032940), we demonstrated that classes of molecules from the above cited applications exhibited biological activity when screened against melanocortin and somatostatin GPCRs. Classes of molecules from the applications WO2004014929 and WO2003082846 were also tested against integrin receptors (Australian patent Application No. 2003900242). Selections of these molecules were also demonstrated to display activity against this class of receptors.

We have now found that libraries of molecules described in the applications WO2004014929 and WO2003082846 can be used to scan molecular diversity. This diversity approach provides an improved method, for effectively identifying selectivity profiles.

It will be clearly understood that, if a prior art publication is referred to herein, this reference does not constitute an admission that the publication forms part of the common general knowledge in the art in Australia or in any other country.

### SUMMARY OF THE INVENTION

In one aspect the invention provides a method of identifying biologically active compounds with defined selectivity profile(s) comprising:
(a) designing a library of compounds of formula I to scan molecular diversity; and
(b) assaying the library of compounds in at least two different biological assays;

wherein formula 1 represents:

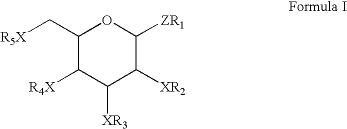

wherein the ring may be of any configuration;

Z is sulphur, oxygen, CH₂, C(O), C(O)NR^{A}, NH, NR^{A} or hydrogen, in the case where Z is hydrogen then R₁ is not present, R^{A} is selected from the set defined for R₁ to R₅, or wherein Z and R1 together form a heterocycle,

X is oxygen or nitrogen, when X is nitrogen, each X may combine independently with the corresponding R₂ to R₅ to form an azide, or wherein each X may also combine independently with any one of corresponding R₂-R₅ to form a heterocycle; R₁ to R₅ are independently selected from the group which includes but is not limited to H or an C1 to C20 alkyl or acyl; C2 to C20 alkenyl, alkynyl, heteroalkyl; C5 to C20 aryl, heteroaryl, arylalkyl or heteroarylalkyl, which is optionally substituted, and can be branched or linear.

In a preferred embodiment the invention relates to a library of compounds selected from compounds of formula 1 when used according to first said method.

In a preferred embodiment, the invention relates to first said method wherein at least one X is nitrogen.

In a preferred embodiment, the invention relates to first said method wherein two of X is nitrogen.

In a preferred embodiment, the invention relates to first said method wherein X and R₂ combine to form heterocycle.

In a preferred embodiment, the invention relates to first said method wherein R₁-R₅ optional substituents are selected from OH, NO, NO₂, NH₂, N₃, halogen, CF₃, CHF₂, CH₂F, nitrile, alkoxy, aryloxy, amidine, guanidiniums, carboxylic acid, carboxylic acid ester, carboxylic acid amide, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoaryl, aminoheteroaryl, thioalkyl, thioaryl or thioheteroaryl, which may optionally be further substituted.

The term “halogen” denotes fluorine, chlorine, bromine or iodine, preferably fluorine, chlorine or bromine.

The term “alkyl” used either alone or in compound words such as “optionally substituted alkyl”, “optionally substituted cycloalkyl”, “arylalkyl” or “heteroarylalkyl”, denotes straight chain, branched or cyclic alkyl, preferably C1-20 alkyl or cycloalkyl. Examples of straight chain and branched alkyl include methyl, ethyl, propyl, isopropyl, butyl, isobutyl, sec-butyl, tert-butyl, amyl, isoamyl, sec-amyl, 1,2-dimethylpropyl, 1,1-dimethylpropyl, hexyl, 4-methylpentyl, 1-methylpentyl, 2-methylpentyl, 3 methylpentyl, 1,1-dimethylbutyl, 2,2-dimethylbutyl, 3,3 dimethylbutyl, 1,2-dimethylbutyl, 1,3-dimethylbutyl, 1,2,2 trimethylpropyl, 1,1,2-trimethylpropyl, heptyl, 5 methylbexyl, 1-methylhexyl, 2,2-dimethypentyl, 3,3 dimethylpentyl, 4,4-dimethylpentyl, 1,2-dimethylpentyl, 1,3-dimethylpentyl, 1,4-dimethylpentyl, 1,2,3 trimethylbutyl, 1,1,2-trimethylbutyl, 1,1,3-trimethylbutyl, octyl, 6-methylheptyl, 1-methylheptyl, 1,1,3,3 tetramethylbutyl, nonyl, 1-, 2-, 3-, 4-, 5-, 6- or 7-methyloctyl, 1-, 2-, 3-, 4- or 5-ethylheptyl, 1-, 2- or 3 propylhexyl, decyl, 1-, 2-, 3-, 4-, 5-, 6-, 7- or 8 methylnonyl, 1-, 2-, 3-, 4-, 5- or 6-ethyloctyl, 1-, 2-, 3 or 4-propylheptyl, undecyl 1-, 2-, 3-, 4-, 5-, 6-, 7-, 8 or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9 or 10-methylundecyl, 1-, 2-, 3-, 4-, 5-, 6-, 7- or 8 ethyldecyl, 1-, 2-, 3-, 4-, 5- or 6-propylnonyl, 1-, 2-, 3 or 4-butyloctyl, 1-2 pentylheptyl and the like. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like.

The term “alkylene” used either alone or in compound words such as “optionally substituted alkylene” denotes the same groups as “alkyl” defined above except that an additional hydrogen has been removed to form a divalent radical. It will be understood that the optional substituent may be attached to or form part of the alkylene chain.

The term “alkenyl” used either alone or in compound words such as “optionally substituted alkenyl” denotes groups formed from straight chain, branched or cyclic alkenes including ethylenically mono-, di- or polyunsaturated alkyl or cycloalkyl groups as defined above, preferably C2-6 alkenyl. Examples of alkenyl include vinyl, allyl, 1-methylvinyl, butenyl, iso-butenyl, 3-methyl-2 butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3 cyclopentadienyl, 1,3-hexadienyl, 1,4-hexadienyl, 1,3 cyclohexadienyl, 1,4-cyclohexadienyl, 1,3-cycloheptadienyl, 1,3,5-cycloheptatrienyl and 1,3,5,7-cyclooctatetraenyl.

The term “alkynyl” used either alone or in compound words, such as “optionally substituted alkynyl” denotes groups formed from straight chain, branched, or mono- or poly- or cyclic alkynes, preferably C2-6 alkynyl.

Examples of alkynyl include ethynyl, 1-propynyl, 1-and 2 butynyl, 2-methyl-2-propynyl, 2-pentynyl, 3-pentynyl, 4pentynyl, 2-hexynyl, 3-hexylnyl, 4-hexynyl, 5-hexynyl, 10 undecynyl, 4-ethyl-I-octyn-3-yl, 7-dodecynyl, 9-dodecynyl, 10-dodecynyl, 3-methyl-1-dodecyn-3-yl, 2-tridecynyl, 11-tridecynyl, 3-tetradecynyl, 7-hexadecynyl, 3-octadecynyl and the like.

The term “alkoxy” used either alone or in compound words such as “optionally substituted alkoxy” denotes straight chain or branched alkoxy, preferably C I-7 alkoxy. Examples of alkoxy include methoxy, ethoxy, npropyloxy, isopropyloxy and the different butoxy isomers.

The term “aryloxy” used either alone or in compound words such as “optionally substituted aryloxy” denotes aromatic, heteroaromatic, arylalkoxy or heteroaryl alkoxy, preferably C6-13 aryloxy. Examples of aryloxy include phenoxy, benzyloxy, 1-napthyloxy, and 2-napthyloxy.

The term “acyl” used either alone or in compound words such as “optionally substituted acyl” or “heteroarylacyl” denotes carbamoyl, aliphatic acyl group and acyl group containing an aromatic ring, which is referred to as aromatic acyl or a heterocyclic ring which is referred to as heterocyclic acyl. Examples of acyl include carbamoyl; straight chain or branched alkanoyl such as formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl, and icosanoyl; alkoxycarbonyl such as methoxycarbonyl, ethoxycarbonyl, t butoxycarbonyl, t-pentyloxycarbonyl and heptyloxycarbonyl; cycloalkylcarbonyl such as cyclopropylcarbonyl cyclobutylcarbonyl, cyclopentylcarbonyl and cyclohexylcarbonyl; alkylsulfonyl such as methylsulfonyl and ethylsulfonyl; alkoxysulfonyl such as methoxysulfonyl and ethoxysulfonyl; aroyl such as benzoyl, toluoyl and naphthoyl; aralkanoyl such as phenylalkanoyl (e. g. phenylacetyl, phenylpropanoyl, phenylbutanoyl, phenylisobutyl, phenylpentanoyl and phenylhexanoyl) and naphthylalkanoyl (e. g. naphthylacetyl, naphthlpropanoyl and naphthylbutanoyl); aralkenoyl such as phenylalkenoyl (e. g. phenylpropenoyl, phenylbutenoyl, phenylmethacrylyl, phenylpentenoyl and phenylhexenoyl and naphthylalkenoyl (e. g. naphthylpropenoyl, naphthylbutenoyl and naphthylpentenoyl); aralkoxycarbonyl such as phenylalkoxycarbonyl (e. g. benzyloxycarbonyl); aryloxycarbonyl such as phenoxycarbonyl and naphthyloxycarbonyl; aryloxyalkanoyl such as phenoxyacetyl and phenoxypropionyl; arylcarbamoyl such as phenylcarbamoyl; arylthiocarbamoyl such as phenylthiocarbamoyl; arylglyoxyloyl such as phenylglyoxyloyl and naphthylglyoxyloyl; arylsulfonyl such as phenylsulfonyl and naphthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolylglyoxyloyl and thienyglyoxyloyl.

The term “aryl” used either alone or in compound words such as “optionally substituted aryl”, “arylalkyl” or “heteroaryl” denotes single, polynuclear, conjugated and fused residues of aromatic hydrocarbons or aromatic heterocyclic ring systems. Examples of aryl include phenyl, biphenyl, terphenyl, quaterphenyl, phenoxyphenyl, naphthyl, tetrahydronaphthyl, anthracenyl, dihydroanthracenyl, benzanthracenyl, dibenzanthracenyl, phenanthrenyl, fluorenyl, pyrenyl, indenyl, azulenyl, chrysenyl, pyridyl, 4-phenylpyridyl, 3-phenylpyridyl, thienyl, furyl, pyrryl, pyrrolyl, furanyl, imadazolyl, pyrrolydinyl, pyridinyl, piperidinyl, indolyl, pyridazinyl, pyrazolyl, pyrazinyl, thiazolyl, pyrimidinyl, quinolinyl, isoquinolinyl, benzofuranyl, benzothienyl, purinyl, quinazolinyl, phenazinyl, acridinyl, benzoxazolyl, benzothiazolyl and the like. Preferably, the aromatic heterocyclic ring system contains 1 to 4 heteroatoms independently selected from N, O and S and containing up to 9 carbon atoms in the ring.

The term “heterocycle” used either alone or in compound words as “optionally substituted heterocycle” denotes monocyclic or polycyclic heterocyclyl groups containing at least one heteroatom atom selected from nitrogen, sulphur and oxygen. Suitable heterocyclyl groups include N-containing heterocyclic groups, such as, unsaturated 3 to 6 membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl or tetrazolyl; saturated to 3 to 6-membered heteromonocyclic groups containing 1 to 4 nitrogen atoms, such as, pyrrolidinyl,imidazolidinyl, piperidin or piperazinyl; unsaturated condensed heterocyclic groups containing 1 to 5 nitrogen atoms, such as, indolyl, isoindolyl, indolizinyl, benzimidazoyl, quinolyl, isoquinolyl, indazolyl, benzotriazolyl or tetrazolopyridazinyl; unsaturated 3 to 6-membered heteromonocyclic group containing an oxygen atom, such as, pyranyl or furyl; unsaturated 3 to 6-membered heteromonocyclic group containing 1 to 2 sulphur atoms, such as, thienyl; unsaturated 3 to 6-membered heteromonocyclic group containing 1 to 2 oxygen atoms and 1 to 3 nitrogen atoms, such as, oxazolyl, isoxazolyl or oxadiazolyl; saturated 3 to 6-membered heteromonocyclic group containing 1 to 2 oxygen atoms and 1 to 3 nitrogen atoms, such as, morpholinyl; unsaturated condensed heterocyclic group containing 1 to 2 oxygen atoms and 1 to 3 nitrogen atoms, such as, benzoxazolyl or benzoxadiazolyl; unsaturated 3 to 6-membered heteromonocyclic group containing 1 to 2 sulphur atoms and 1 to 3 nitrogen atoms, such as, thiazolyl or thiadiazolyl; saturated 3 to 6-membered heteromonocyclic group containing 1 to 2 sulphur atoms and 1 to 3 nitrogen atoms, such as thiazolidinyl; and unsaturated condensed heterocyclic group containing 1 to 2 sulphur atoms and 1 to 3 nitrogen atoms, such as, benzothiazolyl or benzothiadiazolyl.

In a preferred embodiment, the invention relates to first said method comprising a library of compounds selected from compounds of formula II,

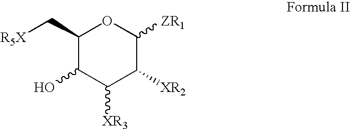

wherein R₁, R₂, R₃, R₅, Z and X are defined as in Formula I.

In a preferred embodiment the invention relates to a library of compounds selected from compounds of formula II.

In a preferred embodiment, the invention relates to first said method comprising a library of compounds selected from compounds of formula III,

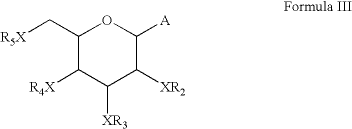

wherein A is defined as hydrogen, SR₁, or OR₁ where R₁ is defined as in Formula I,

and

X and R₂ to R₅ are defined as in Formula I.

In a preferred embodiment the invention relates to a library of compounds selected from compounds of formula III.

In a preferred embodiment, the invention relates to first said method comprising a library of compounds selected from compounds of formula IV,

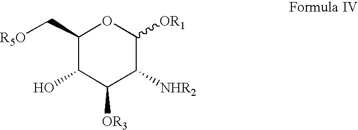

wherein R₁, R₂, R₃ and R₅ are defined as in Formula I.

In a preferred embodiment the invention relates to a library of compounds selected from compounds of f formula IV.

In a preferred embodiment, the invention relates to first said method comprising a library of compounds selected from compounds of formula V,

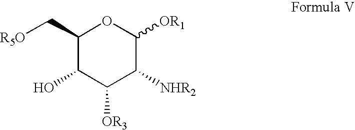

wherein R₁, R₂, R₃ and R₅ are defined as in Formula I.

In a preferred embodiment the invention relates to a library of compounds selected from compounds of formula V.

In a preferred embodiment, the invention relates to first said method comprising a library of compounds selected from compounds of formula VI,

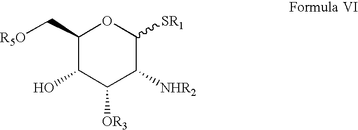

wherein R₁, R₂, R₃ and R₅ are defined as in Formula I.

In a preferred embodiment the invention relates to a library of compounds selected from compounds of formula VI.

In a preferred embodiment, the invention relates to first said method comprising a library of compounds selected from compounds of formula VII,

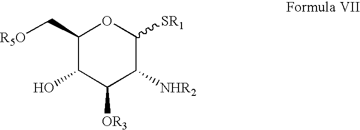

wherein R₁, R₂, R₃ and R₅ are defined as in Formula I.

In a preferred embodiment the invention relates to a library of compounds selected from compounds of formula VII.

In a preferred embodiment, the invention relates to first said method comprising a library of compounds selected from compounds of formula VIII,

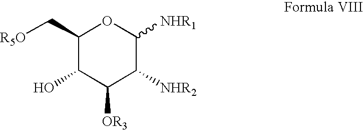

wherein R₁, R₂, R₃ and R₅ are defined as in Formula I.

In a preferred embodiment the invention relates to a library of compounds selected from compounds of formula VIII.

In a preferred embodiment, the invention relates to first said method comprising a library of compounds selected from compounds of formula IX,

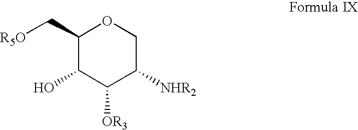

wherein R₂, R₃ and R₅ are defined as in Formula I.

In a preferred embodiment the invention relates to a library of compounds selected from compounds of formula IX.

In a preferred embodiment, the invention relates to said methods wherein biological assays involve Peptide Ligand class of GPCRs.

In a preferred embodiment, the invention relates to first said method wherein biological assays involve opioid, melanocortin, melanin-concentrating hormone, neurokinin, neuropeptide and urotensin receptors.

In a preferred embodiment, the invention relates to first said method wherein biological assays involve δ-opioid (DOP), κ-Opioid (KOP), Melanocortin MC3, Melanocortin MC4, Melanocortin MC5, Melanin-Concentrating Hormone (MCH1), μ-opioid (MOP), Neurokinin (NK1), Neuropeptide Y (NPY-Y1), Opioid (ORL1) and urotensin (UR2) receptors.

In another aspect the invention provides a compound according to formula 1 in which at least one X is nitrogen, and said X is combined with the corresponding R₂-R₅ to form a heterocycle.

In a preferred embodiment, the invention provides a compound according to formula 1 wherein X and R₂ combine to form a heterocycle.

In a preferred embodiment, the invention provides a compound according to formula 1 wherein the heterocycle is heteroaryl, including triazoles, benzimidazoles, benzimidazolone, benzimidazolothione, imidazole, hydantoine, thiohydantoine and purine

### DETAILED DESCRIPTION OF THE INVENTION

Embodiments of the invention will be described with reference to the following examples. Where appropriate, the following abbreviations are used.

Ac Acetyl

DTPM 5-Acyl-1,3-dimethylbarbiturate

Ph Phenyl

TBDMS t-Butyldimethylsilyl

TBDPS t-Butyldiphenylsilyl

Bn benzyl

Bz benzoyl

Me methyl

DCE 1,2-dichloroethane

DCM dichloromethane, methylene chloride

Tf trifluoromethanesulfonyl

Ts 4-methylphenylsulfonyl, p-toluenesulfonyl

DMF N,N-dimethylformamide

DMAP N,N-dimethylaminopyridine

α,α-DMT α,α-dimethoxytoluene, benzaldehyde dimethyl acetal

DMSO dimethylsulfoxide

DTT dithiothreitol

DMTST Dimethyl(methylthio)sulphoniumtrifluoro-methanesulphonate

TBAF tetra-n-butylammonium fluoride

Selectivity profiles are determined by biological assays, either in vitro or in vivo, in which compounds exhibit a specific response in each assay. The panel of specific responses represents the selectivity profile across the selected assays. The selectivity profile may be determined by testing compounds against (a) a series of commercially available assays, and/or (b) self-designed assays. The profile distinguishes actives against non-actives in each assay, as indicated in Table 3 below.

The designing of libraries is based on methods known in the art, including designing to scan for molecular diversity using molecular modeling. The libraries may be designed by using molecular modeling techniques as described by Thanh Le et al (Drug Discovery Today 8, 701-709 (2003)).

Part A: Preparation of Building Blocks:

In order to fully enable the invention, we detail below methods for the preparation of certain building blocks used in the preparation of the compounds of the invention. The building blocks described are suitable for both solution and solid phase synthesis of the compounds of the invention.

Compounds of the library as presented exhibit different selectivity profiles. It is also apparent from these relationships that new compounds with different selectivity profiles may be designed.

### EXAMPLE A

### Synthesis of a 2,4 dinitrogen containing Galactopyranoside Building Block

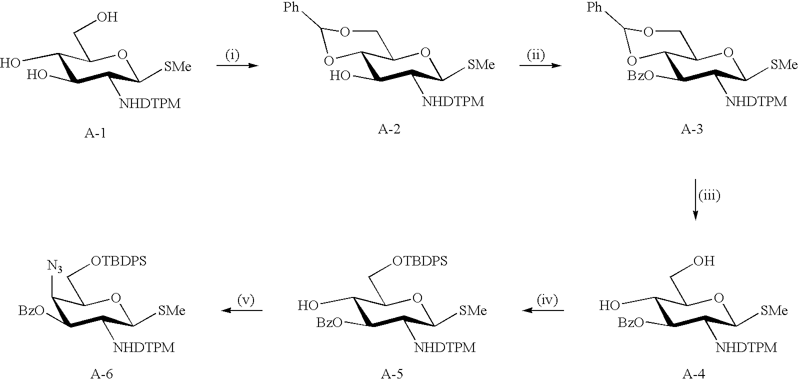

Conditions: (i) α,α-dimethoxytoluene (α,α-DMT), p-toluenesulphonic acid (TsOH), acetonitrile (MeCN), 76° C., 85%; (ii) Benzoylchloride (BzCl), triethylamine; DCM, 99%; (iii) methanol (MeOH)/MeCN/water, TsOH, 75° C., 98%; (iv) t-butyldiphenylsilylchloride (TBDPS-Cl), imidazole, pyridine, 120° C., 99% ; (v) Tf₂O, pyridine, DCM, 0° C., 100%; (b) NaN₃, DMF, 16 hr, RT, 99%.

### EXAMPLE B

### Synthesis of a 3-nitrogen containing Gulopyranoside Building Block

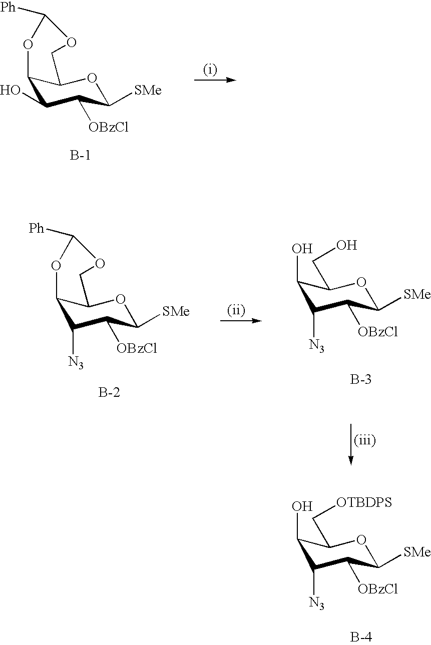

Conditions: (i) (a) trifluoromethanesulfonic anhydride (Tf₂O), pyridine, −20° C., dichloromethane (DCM), 1 hour, 100%, (b) sodium azide (NaN₃), N,N-dimethylformamide (DMF), 50° C., 5 hours, quantitative; (ii) TsOH, MeCN/MeOH/water (12:3:1), 90° C., 6 hours, 88% (iii) TBDPSCl, DMAP, pyridine, 120° C., 12 hours, 93%

### EXAMPLE C

### Synthesis of a 2,6-dinitrogen substituted Glucopyranoside Building Block

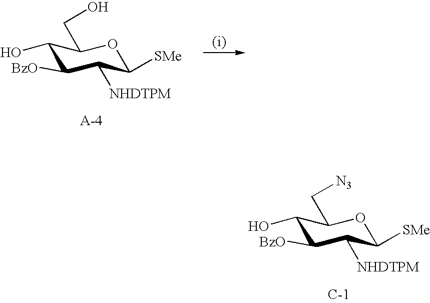

Conditions: (i) (a) Tosylchlodride, pyridine, RT, 24 hours, 33% (b) NaN₃, DMF, RT, 168 hours.

### EXAMPLE D

### Synthesis of a 2-nitrogen containing Tallopyranoside Building Block

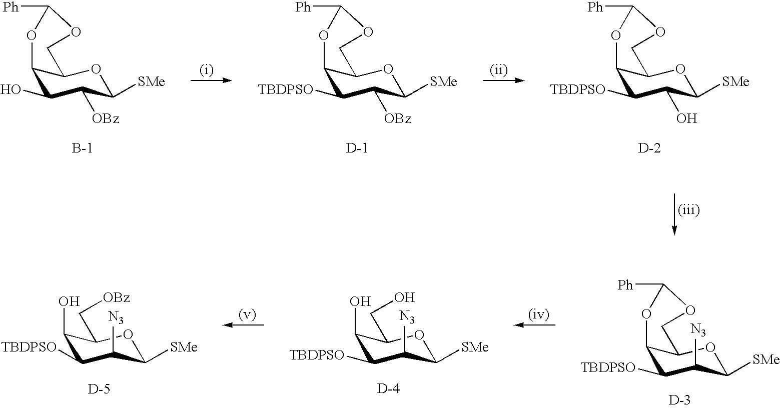

Conditions: (i) TBDPSCl, imidazole, 1,2-DCE, reflux; (ii) NaOMe/MeOH; (iii) (a) Tf₂O, pyridine, −20° C., DCM, 1 hour, (b) NaN₃, DMF, 50° C., 5 hours; (iv) TsOH, MeCN/MeOH/water; (v) benzoylchloride, DMAP, 1,2-DCE, −20° C.

### EXAMPLE E

### Synthesis of two 3-nitrogen containing Altropyranoside Building Block

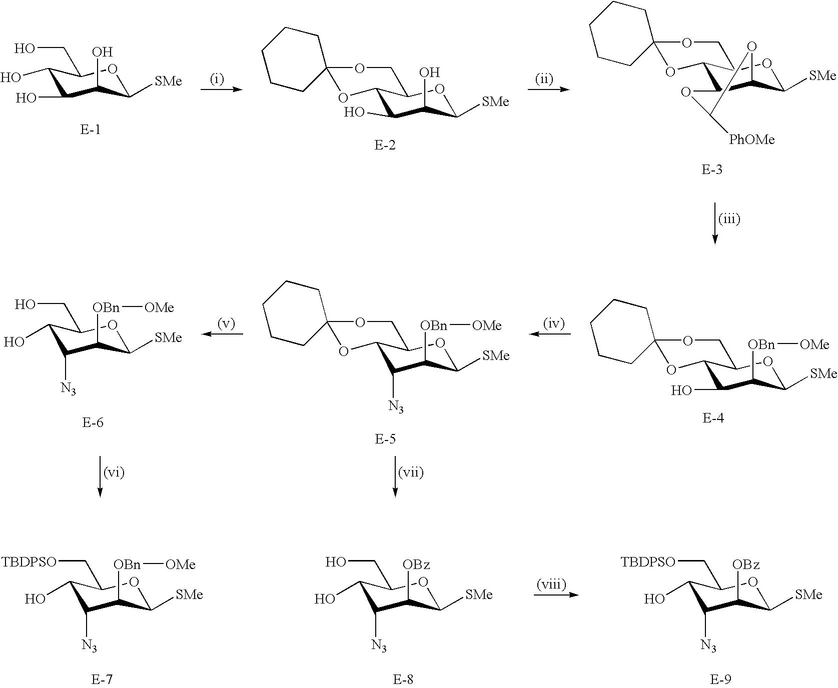

Conditions: (i) cyclohexanone dimethylacetal, TsOH, MeCN; (ii) p-methoxybenzaldehyde dimethylacetal, TsOH, MeCN; (iii) DIBAL, −78° C., diethyl ether; (iv) (a) Tf₂O, pyridine, −20° C., DCM, 1 hour, (b) NaN₃, DMF, 50° C., 5 hours; (v) TsOH, MeCN/MeOH/water; (vi) TBDPSCl, DMAP, 1,2-DCE; (vii) (a) CAN, (b) BzCl, DMAP, 1,2-DCE, (c) TsOH, MeCN/MeOH/water; (viii) TBDPSCl, DMAP, 1,2-DCE.

### EXAMPLE F

### Synthesis of a 2-nitrogen containing Glucopyranoside Building Block

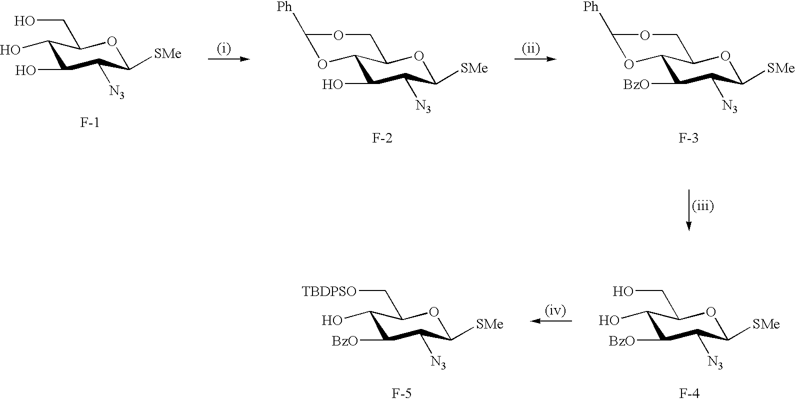

Conditions: (i) α,α-DMT, TsOH, MeCN; (ii) 1,2-DCE, BzCl, DMAP; (iii) TsOH, MeOH/MeCN; (iv) TBDPS-Cl, DMAP, 1,2-DCE.

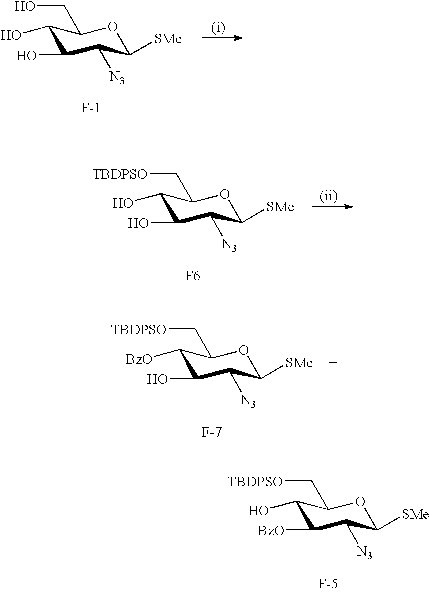

Conditions: (i) TBDPSCl, DMAP, pyridine, 120° C., 0.5 hours, 81%; (ii) a. (Bu)2SnO, MeOH; b. Benzoylchloride, RT, 24 hour;

### EXAMPLE G

### Synthesis of a 2-nitrogen containing Allopyranoside Building Block

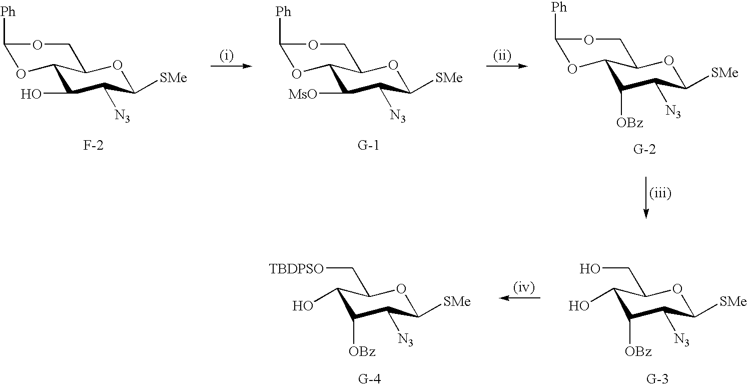

Conditions: (i) DCM/pyridine, MsCl, DMAP, 0° C.; (ii) sodium benzoate, dimethylsulphoxide (DMSO), 140° C.; (iii) TsOH, MeOH/MeCN/water; (iv) TBDPS-Cl, imidazole, DCM, 1 hour, reflux.

Part B: Biological Assays Experimental Method

Cloned receptor membrane preparations from Perkin Elmer Biosignal™ were used in radioligand binding assays.

Membranes (A1-A11=Codes for Table 3: Results).

A1 Human δ-opioid (DOP), A2 Human κ-Opioid (KOP), A3 Human Melanocortin (MC3), A4 Human Melanocortin (MC4), A5 Human melanocortin (MC5), A6 Human melanin-concentrating hormone (MCH1), A7 Human μ-opioid (MOP), A8 Human neurokinin (NK1), A9 Human neuropeptide Y (NPY-Y1), A10 Human opioid (ORL1) A11 Mouse urotensin (mUR2)

Materials and Methods

Screening experiments were performed in either a 50 μl filtration or 25 μl FlashPlate assay format using the following protocol:

TABLE 1
Assay format, radioligands and reference ligands

| | | | Final | | Final |
|---|---|---|---|---|---|
| | Assay | | conc. | Reference | conc. |
| Receptor | format | Radioligand | (nM) | ligand | (μM) |
| MCH1 | 25 μl | [¹²⁵I]-S36057 | 0.1 | MCH | 1 |
| | Flash Plate | | | | |
| MC3 | 50 μl | [¹²⁵I]-NDP- | 0.25 | NDP-αMSH | 10 |
| | Flash Plate | αMSH | | | |
| MC4 | 25 μl | [¹²⁵I]-NDP- | 0.25 | NDP-αMSH | 10 |
| | Flash Plate | αMSH | | | |
| MC5 | 25 μl | [¹²⁵I]-NDP- | 0.25 | NDP-αMSH | 10 |
| | Flash Plate | αMSH | | | |
| NK1 | 50 μl | [¹²⁵I]-Substance P | 0.1 | L703,606 | 10 |
| | Filtration | | | | |
| NPY-Y1 | 25 μl | [¹²⁵I]-PYY | 0.35 | BIBP | 10 |
| | Flash Plate | | | | |
| ORL1 | 25 μl | [¹²⁵I]-Nociceptin | 0.22 | Nociceptin | 1 |
| | Flash Plate | | | | |
| μ-opioid | 25 μl | [³H]-Naloxone | 3 | Naltrexone | 10 |
| | Flash Plate | | | | |
| κ-opioid | 50 μl | [³H]- | 1 | nor-BNI | 1 |
| | Filtration | Diprenorphine | | | |
| δ-opioid | 25 μl | [³H]- | 3 | Naltrindole | 1 |
| | Flash Plate | Bremazocine | | | |
| UR2 | 25 μl | [¹²⁵I]-Urotensin | 0.3 | Urotensin II | 10 |
| | Flash Plate | II | | | |

TABLE 2
Assay buffers

| Receptor | Buffer |
|---|---|
| MCH1 | 25 mM Hepes pH 7.0, 10 mM MgCl₂, 1 mM EDTA and |
| | 0.5% BSA |
| MC3 | 25 mM Tris-HCl pH 7.4, 1 mM MgCl₂, 1.5 mM CaCl₂, |
| | 1 mM NaCl and 0.2% BSA |
| MC4 | 25 mM Tris-HCl pH 7.4, 1 mM MgCl₂, 1.5 mM CaCl₂, |
| | 1 mM NaCl and 0.2% BSA |
| MC5 | 25 mM Tris-HCl pH 7.4, 1 mM MgCl₂, 1.5 mM CaCl₂, |
| | 1 mM NaCl and 0.2% BSA |
| NK1 | 40 mM Hepes pH 7.4, 5 mM MgCl₂, 1 mM EDTA, 0.5% |
| | BSA, 0.025% bacitracin and 25 μM phosphoramidon |
| NPY-Y1 | 50 mM Tris-HCl pH 7.4, 5 mM KCl, 1 mM MgCl₂, |
| | 2 mM CaCl₂, 120 mM NaCl, 0.5% BSA and |
| | 50 μM thiorphan |
| ORL1 | 50 mM Tris-HCl pH 7.4, 10 mM MgCl₂, 1 mM EDTA |
| | and 0.5% BSA |
| μ-opioid | 50 mM Tris-HCl pH 7.4, 10 mM MgCl₂, 1 mM EDTA, |
| | 0.5% BSA and 0.01% bacitracin |
| κ-opioid | 50 mM Tris-HCl pH 7.4 |
| δ-opioid | 50 mM Tris-HCl pH 7.4, 10 mM MgCl₂, 1 mM |
| | EDTA and 0.5% BSA |
| UR2 | 50 mM Tris-HCl pH 7.4, 10 mM MgCl₂, 1 mM |
| | EDTA and 0.5% BSA |

### Format 1: FlashPlate Assay Volumes

19.5 μl buffer, 0.5 μl of compound diluted in DMSO, 5 μl of radioligand diluted in binding buffer.

### Format 2: Filtration Assay Volumes

44 μl membranes diluted in buffer, 1 μl of compound diluted in DMSO, 5 μl of radioligand diluted in binding buffer.

Compound Handling and Dilutions

The day prior to performing the experiment 50 μl DMSO was added to each well of the compound plates to yield compounds at a final concentration of 10 mM. Daughter plates were then created by diluting the compounds further in DMSO to a concentration of 0.5 mM. The mother plates were frozen immediately.

Protocols:

Filtration

Thaw membranes on ice then dilute membranes in binding buffer at a concentration of 1 Unit per well. Dilute radio-ligand to 10 times the final concentration in binding buffer. Add 44 μl of diluted membranes to each well of the deep-well plate. Add 1 μl of DMSO (total value, 5 wells), reference ligand (non-specific value, 3 wells) or compound to the corresponding wells in the deep-well plate. Initiate the reaction by adding 5 μl of radioligand to each well and vortex gently. Incubate at room temperature for 1 hour. During incubation, pre-incubate the Multiscreen Harvest plates in 0.3% PEI. Filter over pre-soaked Multiscreen Harvest plates using a Tomtec Harvester. Wash 9 times with 500 μl of cold 50 mM Tris-HCl pH 7.4 at 4° C. and air-dry for 30 minutes at room temperature under a fume hood. Apply a bottom seal to the Multiscreen Harvest plates. Add 25 μl of MicroScint-0 to each well. Apply TopSeal-A to the plate. Count for 30 seconds per well on TopCount Microplate Scintillation and Luminescence Counter (PerkinElmer) using a count delay of 60 seconds.

FlashPlate

Immobilize membranes into FlashPlate microplates using PerkinElmer BioSignal's proprietary coating procedure. Dilute radioligand to 5× the final concentration in binding buffer. Add 19.5 μl buffer to each well of the FlashPlate. Add 0.5 μl of DMSO (total value, 5 wells), reference ligand (non-specific value, 3 wells) or compound to the corresponding wells in the FlashPlate microplate. Initiate the reaction by adding 5 μl of radioligand to each well. Apply TopSeal-A onto FlashPlate microplates. Incubate at room temperature for 1 hour in the dark. Count for 30 seconds per well on TopCount Microplate Scintillation and Luminescence Counter (PerkinElmer) using a count delay of 60 seconds.

Data Analysis

Percentage inhibition was calculated using the following formula:

$$\% inhibition = \frac{( compound - Total ) \times 100}{Non Specific - Total}$$

TABLE 3
Radioligand Binding Results

| NO | BLOCK | R1 | R2 | R3 | R6 | A1 | A2 | A3 | A4 | A5 | A6 | A7 | A8 | A9 | A10 | A11 |
|---|---|---|---|---|---|---|---|---|---|---|---|---|---|---|---|---|
| 1 | A | X1 | X14 | X1 | X24 | − | − | + | + | + | + | − | + | − | − | − |
| 2 | A | X1 | X15 | X1 | X24 | − | − | − | + | + | + | − | + | − | − | − |
| 3 | A | X1 | X14 | X2 | X24 | − | − | − | − | + | + | − | + | − | − | − |
| 4 | A | X1 | X16 | X2 | X24 | − | − | − | − | + | + | − | + | − | − | − |
| 5 | A | X1 | X15 | X2 | X24 | − | − | − | − | + | − | − | + | − | − | − |
| 6 | A | X1 | X17 | X2 | X24 | − | + | − | − | + | + | + | + | − | − | − |
| 7 | A | X1 | X16 | X8 | X24 | − | − | − | − | − | + | + | + | − | − | − |
| 8 | A | X1 | X17 | X8 | X24 | − | − | − | − | − | + | − | + | − | − | − |
| 9 | A | X1 | X17 | X8 | X24 | − | − | − | − | − | − | − | + | − | − | − |
| 10 | A | X1 | X14 | X3 | X24 | − | − | − | − | + | + | − | + | − | − | − |
| 11 | A | X1 | X16 | X3 | X24 | + | + | − | + | + | + | + | + | − | − | − |
| 12 | A | X1 | X15 | X3 | X24 | − | − | − | − | − | − | − | + | − | − | − |
| 13 | A | X1 | X17 | X3 | X24 | − | − | − | − | + | + | + | + | − | − | − |
| 14 | A | X2 | X14 | X1 | X24 | − | − | − | + | + | + | + | − | − | − | − |
| 15 | A | X2 | X14 | X1 | X24 | − | − | − | + | + | + | + | − | − | − | − |
| 16 | A | X2 | X16 | X1 | X24 | + | + | + | + | + | + | + | + | − | − | − |
| 17 | A | X2 | X14 | X2 | X24 | − | − | − | + | + | + | + | + | − | − | − |
| 18 | A | X2 | X16 | X2 | X24 | − | − | − | − | + | + | + | + | − | − | − |
| 19 | A | X2 | X16 | X8 | X24 | − | − | − | − | − | − | + | + | − | − | − |
| 20 | A | X2 | X15 | X8 | X24 | − | − | − | − | − | − | − | + | − | − | − |
| 21 | A | X2 | X17 | X8 | X24 | − | − | − | − | − | − | − | + | − | − | − |
| 22 | A | X2 | X14 | X3 | X24 | − | − | − | − | + | − | − | − | − | − | − |
| 23 | A | X8 | X14 | X1 | X24 | − | − | − | − | + | + | − | + | − | − | − |
| 24 | A | X8 | X16 | X1 | X24 | − | − | − | − | + | + | − | + | − | − | − |
| 25 | A | X8 | X17 | X1 | X24 | − | − | − | + | + | + | − | + | − | − | − |
| 26 | A | X8 | X15 | X2 | X24 | − | − | − | − | − | − | − | + | − | − | − |
| 27 | A | X8 | X14 | X3 | X24 | − | − | − | − | + | − | − | − | − | − | − |
| 28 | A | X3 | X14 | X1 | X24 | − | − | − | + | + | + | − | + | − | − | − |
| 29 | A | X3 | X16 | X1 | X24 | − | − | + | + | + | + | − | + | − | − | − |
| 30 | A | X3 | X17 | X1 | X24 | − | − | + | + | + | + | + | + | − | − | − |
| 31 | A | X3 | X14 | X2 | X24 | − | − | + | − | + | − | + | − | − | − | − |
| 32 | A | X3 | X16 | X2 | X24 | + | − | − | − | + | − | − | + | − | − | − |
| 33 | A | X3 | X17 | X2 | X24 | − | − | − | − | − | + | − | + | − | − | − |
| 34 | A | X3 | X16 | X8 | X24 | − | − | − | − | − | − | − | + | − | − | − |
| 35 | A | X3 | X15 | X8 | X24 | − | − | − | − | − | − | − | + | − | − | − |
| 36 | A | X3 | X17 | X8 | X24 | − | − | − | − | − | − | + | + | − | − | − |
| 37 | A | X3 | X14 | X3 | X24 | − | − | + | + | + | + | − | − | − | − | − |
| 38 | A | X3 | X16 | X3 | X24 | − | − | − | − | + | + | − | + | − | − | − |
| 39 | A | X3 | X17 | X3 | X24 | − | − | − | − | − | + | − | + | − | − | − |
| 40 | A | X7 | X14 | X1 | X24 | − | − | + | + | + | + | − | − | − | − | − |
| 41 | A | X7 | X16 | X1 | X24 | − | − | − | + | + | + | − | + | − | − | − |
| 42 | A | X7 | X15 | X1 | X24 | − | − | − | − | + | − | − | − | − | − | − |
| 43 | A | X7 | X17 | X1 | X24 | − | + | + | + | + | + | + | + | − | − | − |
| 44 | A | X7 | X14 | X2 | X24 | − | − | − | + | + | − | − | − | − | − | − |
| 45 | A | X7 | X16 | X2 | X24 | − | − | − | + | − | − | − | + | − | − | − |
| 46 | A | X7 | X16 | X8 | X24 | − | − | − | − | − | − | − | + | − | − | − |
| 47 | A | X7 | X15 | X8 | X24 | − | − | − | − | − | − | − | + | − | − | − |
| 48 | A | X7 | X14 | X3 | X24 | − | − | − | + | + | + | − | − | − | − | − |
| 49 | A | X7 | X16 | X3 | X24 | − | − | − | + | − | − | − | + | − | − | − |
| 50 | A | X7 | X17 | X3 | X24 | − | − | − | − | − | + | − | + | − | − | − |
| 51 | A | X1 | X14 | X24 | X1 | − | − | + | + | + | + | − | + | − | + | − |
| 52 | A | X1 | X16 | X24 | X1 | − | − | + | + | + | + | + | + | − | + | − |
| 53 | A | X1 | X15 | X24 | X1 | − | − | − | − | − | + | − | − | − | − | − |
| 54 | A | X1 | X17 | X24 | X1 | − | − | + | + | + | + | + | + | − | + | − |
| 55 | A | X1 | X14 | X24 | X2 | − | + | + | + | + | + | + | + | − | + | − |
| 56 | A | X1 | X16 | X24 | X2 | − | − | − | − | + | − | + | + | − | − | − |
| 57 | A | X1 | X17 | X24 | X2 | − | + | + | + | + | − | + | + | − | + | − |
| 58 | A | X1 | X14 | X24 | X8 | − | + | − | + | + | − | + | + | − | − | − |
| 59 | A | X1 | X16 | X24 | X8 | − | + | − | − | + | − | + | + | − | − | − |
| 60 | A | X1 | X15 | X24 | X8 | − | − | − | − | + | − | − | − | − | − | − |
| 61 | A | X1 | X17 | X24 | X8 | − | + | − | − | + | − | + | + | − | − | − |
| 62 | A | X1 | X14 | X24 | X3 | − | − | + | + | + | + | − | + | − | + | − |
| 63 | A | X1 | X16 | X24 | X3 | − | + | + | + | + | + | + | + | + | + | − |
| 64 | A | X1 | X15 | X24 | X3 | − | − | + | − | + | − | − | + | − | − | − |
| 65 | A | X1 | X17 | X24 | X3 | − | − | + | + | + | − | + | + | − | − | − |
| 66 | A | X2 | X14 | X24 | X1 | − | − | + | + | + | + | + | + | − | − | − |
| 67 | A | X2 | X16 | X24 | X1 | + | − | + | + | + | + | + | + | − | + | − |
| 68 | A | X2 | X15 | X24 | X1 | − | − | + | + | + | + | − | + | − | − | − |
| 69 | A | X2 | X17 | X24 | X1 | − | − | + | + | + | + | + | + | − | + | − |
| 70 | A | X2 | X14 | X24 | X2 | − | − | + | + | + | − | + | + | − | − | − |
| 71 | A | X2 | X16 | X24 | X2 | − | + | + | + | + | + | + | + | − | + | − |
| 72 | A | X2 | X15 | X24 | X2 | − | − | − | + | + | − | − | + | − | − | − |
| 73 | A | X2 | X17 | X24 | X2 | − | + | − | − | + | − | + | + | − | + | − |
| 74 | A | X2 | X14 | X24 | X8 | − | − | − | − | + | − | + | + | − | − | − |
| 75 | A | X2 | X16 | X24 | X8 | − | − | − | − | − | − | + | + | − | − | − |
| 76 | A | X2 | X15 | X24 | X8 | − | − | − | − | − | − | − | + | − | − | − |
| 77 | A | X2 | X17 | X24 | X8 | − | − | − | − | + | − | + | + | − | − | − |
| 78 | A | X2 | X14 | X24 | X3 | − | − | − | − | + | − | − | + | − | − | − |
| 79 | A | X2 | X16 | X24 | X3 | − | − | − | + | + | − | + | + | − | − | − |
| 80 | A | X2 | X15 | X24 | X3 | − | − | − | + | + | − | − | + | − | − | − |
| 81 | A | X2 | X17 | X24 | X3 | − | + | + | + | + | − | + | + | − | − | − |
| 82 | A | X8 | X14 | X24 | X1 | − | − | − | + | − | − | − | + | − | − | − |
| 83 | A | X8 | X16 | X24 | X1 | − | − | − | − | + | − | + | + | − | − | − |
| 84 | A | X8 | X15 | X24 | X1 | − | − | − | − | − | − | − | + | − | − | − |
| 85 | A | X8 | X17 | X24 | X1 | − | − | + | + | + | + | + | + | − | + | − |
| 86 | A | X8 | X14 | X24 | X2 | − | − | + | + | + | − | + | + | − | − | − |
| 87 | A | X8 | X16 | X24 | X2 | − | − | − | − | − | − | − | + | − | − | − |
| 88 | A | X8 | X15 | X24 | X2 | − | − | − | − | − | − | − | + | − | − | − |
| 89 | A | X8 | X17 | X24 | X2 | − | − | − | − | − | − | + | + | − | − | − |
| 90 | A | X8 | X14 | X24 | X3 | − | − | − | − | − | − | − | + | − | − | − |
| 91 | A | X8 | X16 | X24 | X3 | − | − | − | − | − | − | − | + | − | − | − |
| 92 | A | X8 | X15 | X24 | X3 | − | − | − | − | − | − | − | + | − | − | − |
| 93 | A | X8 | X17 | X24 | X3 | − | − | − | − | + | − | + | + | − | − | − |
| 94 | A | X3 | X16 | X24 | X1 | − | − | + | + | + | + | − | + | + | + | − |
| 95 | A | X3 | X15 | X24 | X1 | − | − | − | + | + | + | − | + | − | + | − |
| 96 | A | X3 | X17 | X24 | X1 | − | + | + | + | + | + | − | + | + | + | − |
| 97 | A | X3 | X14 | X24 | X2 | − | − | + | + | + | − | − | + | − | + | − |
| 98 | A | X3 | X16 | X24 | X2 | − | − | − | + | + | + | − | + | − | − | − |
| 99 | A | X3 | X15 | X24 | X2 | − | − | − | + | + | − | − | + | − | − | − |
| 100 | A | X3 | X17 | X24 | X2 | − | − | + | + | + | + | + | + | − | + | − |
| 101 | A | X3 | X14 | X24 | X8 | − | − | − | − | + | − | − | − | − | − | − |
| 102 | A | X3 | X16 | X24 | X8 | − | − | − | − | + | − | − | − | − | − | − |
| 103 | A | X3 | X15 | X24 | X8 | − | − | − | − | − | − | − | + | − | − | − |
| 104 | A | X3 | X17 | X24 | X8 | − | − | − | + | + | − | + | + | − | − | − |
| 105 | A | X3 | X14 | X24 | X3 | − | − | − | + | + | − | − | + | − | − | − |
| 106 | A | X3 | X16 | X24 | X3 | − | − | + | + | + | − | − | + | − | − | − |
| 107 | A | X3 | X15 | X24 | X3 | − | − | − | + | + | − | − | + | − | − | − |
| 108 | A | X3 | X17 | X24 | X3 | − | − | + | + | + | − | − | + | − | − | − |
| 109 | A | X7 | X14 | X24 | X1 | − | − | + | + | + | − | − | − | − | − | − |
| 110 | A | X7 | X16 | X24 | X1 | − | − | − | + | + | − | − | − | − | − | − |
| 111 | A | X7 | X15 | X24 | X1 | − | − | − | − | − | − | − | + | − | − | − |
| 112 | A | X7 | X17 | X24 | X1 | − | − | + | + | + | − | − | + | − | − | − |
| 113 | A | X7 | X14 | X24 | X2 | − | − | − | + | + | − | − | + | − | + | − |
| 114 | A | X7 | X16 | X24 | X2 | − | + | − | + | + | − | − | + | − | − | − |
| 115 | A | X7 | X15 | X24 | X2 | − | − | − | − | − | − | − | + | − | − | − |
| 116 | A | X7 | X17 | X24 | X2 | − | − | − | − | + | − | − | + | − | − | − |
| 117 | A | X7 | X16 | X24 | X8 | − | + | − | − | − | − | − | + | − | − | − |
| 118 | A | X7 | X17 | X24 | X8 | − | − | − | − | − | − | − | + | − | − | − |
| 119 | A | X7 | X14 | X24 | X3 | − | − | − | − | − | − | − | + | − | − | − |
| 120 | A | X7 | X16 | X24 | X3 | − | + | − | + | + | − | − | + | − | − | − |
| 121 | A | X7 | X15 | X24 | X3 | − | − | − | − | + | − | − | + | − | − | − |
| 122 | A | X7 | X17 | X24 | X3 | − | − | − | + | + | − | − | + | − | − | − |
| 123 | A | X16 | X25 | X2 | X24 | − | − | − | − | + | − | − | + | − | − | − |
| 124 | A | X16 | X25 | X24 | X2 | − | − | − | − | + | − | − | + | − | − | − |
| 125 | A | X16 | X23 | X24 | X8 | − | − | − | − | − | − | − | + | − | − | − |
| 126 | A | X16 | N3 | X8 | X2 | − | − | − | + | + | + | − | + | − | − | − |
| 127 | A | X16 | N3 | X2 | X8 | − | − | − | + | + | − | − | + | − | − | − |
| 128 | A | X24 | X11 | X8 | X26 | − | − | − | − | − | − | − | + | − | − | − |
| 129 | A | X1 | X14 | X4 | X24 | − | − | − | − | − | − | + | − | − | − | − |
| 130 | A | X1 | X16 | X4 | X24 | − | − | − | − | − | − | + | − | − | − | − |
| 131 | A | X1 | X15 | X4 | X24 | − | − | − | − | − | + | P | − | − | − | − |
| 132 | A | X1 | X17 | X4 | X24 | − | + | − | − | − | − | + | − | − | − | − |
| 133 | A | X2 | X14 | X4 | X24 | − | − | − | − | − | − | + | − | − | − | − |
| 134 | A | X2 | X15 | X4 | X24 | − | − | − | − | − | − | + | − | − | − | − |
| 135 | A | X2 | X17 | X4 | X24 | − | − | − | − | − | − | + | − | − | − | − |
| 136 | A | X8 | X15 | X4 | X24 | − | − | − | − | − | − | − | + | − | − | − |
| 137 | A | X3 | X14 | X4 | X24 | − | − | − | − | + | − | + | − | − | − | − |
| 138 | A | X3 | X16 | X4 | X24 | + | − | − | − | − | − | + | − | − | − | − |
| 139 | A | X3 | X17 | X4 | X24 | − | − | − | − | − | + | + | − | − | − | − |
| 140 | A | X7 | X15 | X4 | X24 | − | − | − | − | − | − | − | + | − | − | − |
| 141 | A | X4 | X14 | X1 | X24 | − | − | − | − | + | + | − | − | − | − | − |
| 142 | A | X4 | X16 | X1 | X24 | − | − | − | − | − | + | − | − | − | − | − |
| 143 | A | X4 | X15 | X1 | X24 | − | − | − | − | − | − | + | + | − | − | − |
| 144 | A | X4 | X17 | X1 | X24 | − | − | − | − | + | + | + | + | − | − | − |
| 145 | A | X4 | X16 | X3 | X24 | − | − | − | − | − | + | − | − | − | − | − |
| 146 | A | X1 | X14 | X24 | X4 | − | + | − | − | + | − | − | + | − | − | − |
| 147 | A | X1 | X16 | X24 | X4 | − | + | − | + | + | + | + | + | − | − | − |
| 148 | A | X1 | X15 | X24 | X4 | − | − | − | − | − | − | − | + | − | − | − |
| 149 | A | X1 | X17 | X24 | X4 | − | − | − | − | − | − | + | + | − | − | − |
| 150 | A | X2 | X14 | X24 | X4 | − | + | − | − | + | − | + | − | − | − | − |
| 151 | A | X2 | X16 | X24 | X4 | − | − | − | − | − | − | + | − | − | − | − |
| 152 | A | X2 | X17 | X24 | X4 | − | − | − | − | − | − | + | − | − | − | − |
| 153 | A | X8 | X15 | X24 | X4 | − | − | − | − | − | − | − | + | − | − | − |
| 154 | A | X8 | X17 | X24 | X4 | − | − | − | − | − | − | − | + | − | − | − |
| 155 | A | X3 | X14 | X24 | X4 | − | − | − | − | + | − | − | − | − | − | − |
| 156 | A | X3 | X15 | X24 | X4 | − | − | − | − | + | − | − | + | − | − | − |
| 157 | A | X3 | X17 | X24 | X4 | − | − | − | − | + | + | − | + | − | − | − |
| 158 | A | X7 | X14 | X24 | X4 | − | − | − | − | − | − | − | + | − | − | − |
| 159 | A | X7 | X16 | X24 | X4 | − | − | − | − | − | − | − | + | − | − | − |
| 160 | A | X7 | X15 | X24 | X4 | − | − | − | − | − | − | − | + | − | − | − |
| 161 | A | X7 | X17 | X24 | X4 | − | − | − | − | − | − | − | + | − | − | − |
| 162 | A | X4 | X14 | X24 | X1 | − | − | − | − | − | − | − | + | − | − | − |
| 163 | A | X4 | X16 | X24 | X1 | − | − | + | + | + | − | − | + | − | − | − |
| 164 | A | X4 | X15 | X24 | X1 | − | − | − | + | − | − | − | + | − | − | − |
| 165 | A | X4 | X14 | X24 | X2 | − | − | − | − | − | − | − | + | − | − | − |
| 166 | A | X4 | X16 | X24 | X2 | − | − | − | + | + | − | − | + | − | − | − |
| 167 | A | X4 | X15 | X24 | X2 | − | − | − | − | − | − | − | + | − | − | − |
| 168 | A | X4 | X17 | X24 | X2 | − | − | − | − | − | − | + | + | − | − | − |
| 169 | A | X4 | X15 | X24 | X3 | − | − | − | − | − | − | − | + | − | − | − |
| 170 | A | X4 | X17 | X24 | X3 | − | − | − | − | + | − | + | + | − | − | − |
| 171 | B | X8 | X14 | X8 | X24 | − | − | − | − | + | − | − | + | − | − | − |
| 172 | B | X8 | X20 | X8 | X24 | − | − | − | − | − | − | − | + | − | − | − |
| 173 | B | X8 | X16 | X8 | X24 | − | − | − | − | − | − | − | + | − | − | − |
| 174 | B | X8 | X15 | X8 | X24 | − | − | − | − | − | − | − | + | − | − | − |
| 175 | B | X8 | X17 | X8 | X24 | − | − | − | − | − | − | − | + | − | − | − |
| 176 | B | X8 | X19 | X3 | X24 | − | − | − | − | − | − | − | + | − | − | − |
| 177 | B | X8 | X14 | X3 | X24 | − | − | + | + | + | − | − | + | − | − | − |
| 178 | B | X8 | X20 | X3 | X24 | − | − | − | − | + | − | − | + | − | − | − |
| 179 | B | X8 | X16 | X3 | X24 | + | − | + | + | + | + | − | + | − | − | − |
| 180 | B | X8 | X15 | X3 | X24 | − | − | − | − | − | − | − | + | − | − | − |
| 181 | B | X8 | X17 | X3 | X24 | − | − | − | + | + | + | − | + | − | − | − |
| 182 | B | X8 | X19 | X1 | X24 | − | − | − | − | − | − | − | + | − | − | − |
| 183 | B | X8 | X14 | X1 | X24 | − | − | − | + | + | + | − | + | − | − | − |
| 184 | B | X8 | X20 | X1 | X24 | − | − | − | − | + | − | − | + | − | − | − |
| 185 | B | X8 | X16 | X1 | X24 | − | − | − | + | + | + | − | + | − | − | − |
| 186 | B | X8 | X15 | X1 | X24 | − | − | − | − | + | − | − | + | − | − | − |
| 187 | B | X8 | X17 | X1 | X24 | − | − | − | + | + | + | − | + | − | − | − |
| 188 | B | X8 | X19 | X2 | X24 | − | − | − | − | − | − | − | + | − | − | − |
| 189 | B | X8 | X14 | X2 | X24 | − | − | + | + | + | + | − | + | − | − | − |
| 190 | B | X8 | X20 | X2 | X24 | − | − | − | − | + | − | − | + | − | − | − |
| 191 | B | X8 | X16 | X2 | X24 | − | − | − | + | + | + | − | + | − | − | − |
| 192 | B | X8 | X15 | X2 | X24 | − | − | − | − | + | − | − | + | − | − | − |
| 193 | B | X8 | X17 | X2 | X24 | − | − | + | + | + | + | − | + | − | − | − |
| 194 | B | X3 | X14 | X8 | X24 | − | − | − | + | + | − | − | + | − | − | − |
| 195 | B | X3 | X20 | X8 | X24 | − | − | − | − | − | − | − | + | − | − | − |
| 196 | B | X3 | X16 | X8 | X24 | + | − | − | − | − | − | − | + | − | − | − |
| 197 | B | X3 | X15 | X8 | X24 | − | − | − | − | − | − | − | + | − | − | − |
| 198 | B | X3 | X17 | X8 | X24 | − | − | − | − | + | − | − | + | − | − | − |
| 199 | B | X3 | X19 | X3 | X24 | − | − | − | − | − | − | − | + | − | − | − |
| 200 | B | X3 | X14 | X3 | X24 | − | − | + | + | + | − | − | + | − | − | − |
| 201 | B | X3 | X20 | X3 | X24 | − | − | − | − | + | − | − | + | − | − | − |
| 202 | B | X3 | X16 | X3 | X24 | − | − | + | + | + | + | − | + | − | − | − |
| 203 | B | X3 | X15 | X3 | X24 | − | − | − | − | + | − | − | + | − | − | − |
| 204 | B | X3 | X17 | X3 | X24 | − | − | + | + | + | + | − | + | − | − | − |
| 205 | B | X3 | X19 | X1 | X24 | − | − | − | + | + | − | − | + | − | − | − |
| 206 | B | X3 | X16 | X1 | X24 | − | − | − | + | + | + | − | + | − | − | − |
| 207 | B | X3 | X15 | X1 | X24 | − | − | + | − | + | − | − | + | − | − | − |
| 208 | B | X3 | X19 | X2 | X24 | − | − | − | − | + | − | − | + | − | − | − |
| 209 | B | X3 | X14 | X2 | X24 | − | − | + | + | + | − | − | + | − | − | − |
| 210 | B | X3 | X20 | X2 | X24 | − | − | − | + | + | − | − | + | − | − | − |
| 211 | B | X3 | X16 | X2 | X24 | − | + | − | + | + | + | + | + | − | − | − |
| 212 | B | X3 | X15 | X2 | X24 | − | − | − | − | + | − | − | + | − | − | − |
| 213 | B | X3 | X17 | X2 | X24 | − | + | − | + | + | + | − | + | − | + | − |
| 214 | B | X7 | X19 | X8 | X24 | − | − | − | − | − | − | − | + | − | − | − |
| 215 | B | X7 | X14 | X8 | X24 | − | − | − | − | + | − | − | + | − | − | − |
| 216 | B | X7 | X20 | X8 | X24 | − | − | − | − | − | − | − | + | − | − | − |
| 217 | B | X7 | X15 | X8 | X24 | − | − | − | − | − | − | − | + | − | − | − |
| 218 | B | X7 | X17 | X8 | X24 | − | − | − | − | − | − | − | + | − | − | − |
| 219 | B | X7 | X19 | X3 | X24 | − | − | − | − | + | − | − | + | − | − | − |
| 220 | B | X7 | X14 | X3 | X24 | − | − | + | + | + | − | − | + | − | − | − |
| 221 | B | X7 | X20 | X3 | X24 | − | − | − | − | + | − | − | + | − | − | − |
| 222 | B | X7 | X15 | X3 | X24 | − | − | − | − | + | − | − | + | − | − | − |
| 223 | B | X7 | X17 | X3 | X24 | − | − | − | + | + | + | − | + | − | − | − |
| 224 | B | X7 | X19 | X1 | X24 | − | − | − | − | − | − | − | + | − | − | − |
| 225 | B | X7 | X19 | X2 | X24 | − | − | − | − | − | − | − | + | − | − | − |
| 226 | B | X7 | X14 | X2 | X24 | − | − | + | + | + | − | − | + | − | − | − |
| 227 | B | X7 | X20 | X2 | X24 | − | − | − | − | − | − | − | + | − | − | − |
| 228 | B | X7 | X15 | X2 | X24 | − | − | − | − | + | − | − | + | − | − | − |
| 229 | B | X7 | X17 | X2 | X24 | − | − | − | + | + | + | − | + | − | − | − |
| 230 | B | X2 | X14 | X8 | X24 | − | − | − | − | + | − | − | + | − | − | − |
| 231 | B | X2 | X20 | X8 | X24 | − | − | − | − | − | − | − | + | − | − | − |
| 232 | B | X2 | X16 | X8 | X24 | − | − | − | − | − | − | − | + | − | − | − |
| 233 | B | X2 | X15 | X8 | X24 | − | − | − | − | − | − | − | + | − | − | − |
| 234 | B | X2 | X17 | X8 | X24 | − | − | − | − | − | − | − | + | − | − | − |
| 235 | B | X2 | X19 | X3 | X24 | + | − | − | − | − | − | − | + | − | − | − |
| 236 | B | X2 | X14 | X3 | X24 | − | − | + | + | + | + | − | + | − | − | − |
| 237 | B | X2 | X20 | X3 | X24 | − | − | − | − | + | − | − | + | − | − | − |
| 238 | B | X2 | X16 | X3 | X24 | + | − | + | + | + | + | − | + | − | + | − |
| 239 | B | X2 | X15 | X3 | X24 | − | − | − | − | + | − | − | + | − | − | − |
| 240 | B | X2 | X17 | X3 | X24 | − | + | + | + | + | + | + | + | − | + | − |
| 241 | B | X2 | X19 | X1 | X24 | − | − | − | + | + | − | − | + | − | − | − |
| 242 | B | X2 | X14 | X1 | X24 | − | − | + | + | + | − | − | + | − | − | − |
| 243 | B | X2 | X20 | X1 | X24 | − | − | − | + | + | − | − | + | − | − | − |
| 244 | B | X2 | X16 | X1 | X24 | − | − | + | + | + | + | − | + | + | − | − |
| 245 | B | X2 | X15 | X1 | X24 | − | − | − | − | − | − | − | + | − | − | − |
| 246 | B | X2 | X19 | X2 | X24 | − | − | − | − | − | − | − | + | − | − | − |
| 247 | B | X2 | X14 | X2 | X24 | − | − | + | + | + | − | − | + | − | − | − |
| 248 | B | X2 | X20 | X2 | X24 | − | − | − | − | + | − | − | + | − | − | − |
| 249 | B | X2 | X16 | X2 | X24 | − | − | + | + | + | + | + | + | − | − | − |
| 250 | B | X2 | X17 | X2 | X24 | − | − | − | + | + | + | − | + | − | + | − |
| 251 | D | X24 | X16 | X8 | X8 | − | − | − | − | − | − | − | + | − | − | − |
| 252 | D | X24 | X17 | X8 | X8 | − | − | − | − | − | − | − | + | − | − | − |
| 253 | D | X24 | X14 | X8 | X3 | − | − | − | − | + | − | − | + | − | − | − |
| 254 | D | X24 | X16 | X8 | X3 | − | − | − | − | − | − | − | + | − | − | − |
| 255 | D | X24 | X14 | X8 | X1 | − | − | − | + | + | − | − | + | − | − | − |
| 256 | D | X24 | X20 | X8 | X1 | − | − | − | − | − | − | − | + | − | − | − |
| 257 | D | X24 | X16 | X8 | X1 | − | − | − | + | − | − | − | + | − | − | − |
| 258 | D | X24 | X15 | X8 | X1 | − | − | − | − | − | − | − | + | − | − | − |
| 259 | D | X24 | X17 | X8 | X1 | − | − | − | + | − | − | − | + | − | − | − |
| 260 | D | X24 | X14 | X8 | X2 | − | − | − | + | − | − | − | − | − | − | − |
| 261 | D | X24 | X20 | X8 | X2 | − | − | − | − | + | − | − | − | − | − | − |
| 262 | D | X24 | X16 | X8 | X2 | − | − | − | − | + | − | − | + | − | − | − |
| 263 | D | X24 | X15 | X8 | X2 | − | − | − | − | − | − | − | + | − | − | − |
| 264 | D | X24 | X17 | X8 | X2 | − | − | − | − | − | − | − | + | − | − | − |
| 265 | D | X24 | X14 | X3 | X8 | − | − | − | + | − | − | − | + | − | − | − |
| 266 | D | X24 | X20 | X3 | X8 | − | − | − | − | − | − | − | + | − | − | − |
| 267 | D | X24 | X16 | X3 | X8 | − | − | − | + | − | − | − | + | − | − | − |
| 268 | D | X24 | X17 | X3 | X8 | − | − | − | − | + | − | − | + | − | − | − |
| 269 | D | X24 | X19 | X3 | X3 | − | − | − | − | − | − | − | + | − | − | − |
| 270 | D | X24 | X14 | X3 | X3 | − | − | + | + | + | − | − | + | − | − | − |
| 271 | D | X24 | X20 | X3 | X3 | − | − | − | − | − | − | − | + | − | − | − |
| 272 | D | X24 | X16 | X3 | X3 | − | − | − | + | + | − | − | + | − | − | − |
| 273 | D | X24 | X15 | X3 | X3 | − | − | − | − | − | − | − | + | − | − | − |
| 274 | D | X24 | X17 | X3 | X3 | − | − | − | − | − | − | − | + | − | − | − |
| 275 | D | X24 | X14 | X3 | X1 | − | − | − | + | + | − | − | + | − | − | − |
| 276 | D | X24 | X20 | X3 | X1 | − | − | − | − | − | − | − | + | − | − | − |
| 277 | D | X24 | X16 | X3 | X1 | − | − | + | − | − | − | − | + | − | − | − |
| 278 | D | X24 | X15 | X3 | X1 | − | − | − | − | − | − | − | + | − | − | − |
| 279 | D | X24 | X17 | X3 | X1 | − | − | − | − | + | − | − | + | − | − | − |
| 280 | D | X24 | X19 | X3 | X2 | − | − | − | − | − | − | − | + | − | − | − |
| 281 | D | X24 | X14 | X3 | X2 | − | − | + | + | + | − | − | + | − | − | − |
| 282 | D | X24 | X20 | X3 | X2 | − | − | − | + | − | − | − | + | − | − | − |
| 283 | D | X24 | X16 | X3 | X2 | − | − | − | + | + | − | − | + | − | − | − |
| 284 | D | X24 | X14 | X1 | X8 | − | − | − | + | + | − | − | + | − | − | − |
| 285 | D | X24 | X20 | X1 | X8 | − | − | − | − | − | − | − | + | − | − | − |
| 286 | D | X24 | X16 | X1 | X8 | − | − | − | − | + | − | − | + | − | − | − |
| 287 | D | X24 | X17 | X1 | X8 | − | − | − | − | − | − | − | + | − | − | − |
| 288 | D | X24 | X19 | X1 | X3 | − | − | − | − | − | − | − | + | − | − | − |
| 289 | D | X24 | X14 | X1 | X3 | − | − | − | + | + | − | − | + | − | − | − |
| 290 | D | X24 | X20 | X1 | X3 | − | − | − | − | + | − | − | + | − | − | − |
| 291 | D | X24 | X16 | X1 | X3 | − | − | − | + | − | − | − | + | − | − | − |
| 292 | D | X24 | X17 | X1 | X3 | − | − | − | − | + | − | − | + | − | − | − |
| 293 | D | X24 | X16 | X1 | X1 | − | − | − | − | − | − | − | + | − | − | − |
| 294 | D | X24 | X15 | X1 | X1 | − | − | − | − | + | − | − | + | − | − | − |
| 295 | D | X24 | X19 | X1 | X2 | − | − | − | − | − | − | − | + | − | − | − |
| 296 | D | X24 | X14 | X1 | X2 | − | − | − | − | − | − | − | + | − | − | − |
| 297 | D | X24 | X20 | X1 | X2 | − | − | − | − | − | − | − | + | − | − | − |
| 298 | D | X24 | X16 | X1 | X2 | − | − | − | − | + | − | − | + | − | − | − |
| 299 | D | X24 | X15 | X1 | X2 | − | − | − | + | − | − | − | + | − | − | − |
| 300 | D | X24 | X17 | X1 | X2 | − | − | − | − | + | − | − | + | − | − | − |
| 301 | D | X24 | X19 | X2 | X8 | − | − | − | − | − | − | − | + | − | − | − |
| 302 | D | X24 | X14 | X2 | X8 | − | − | − | + | + | − | − | + | − | − | − |
| 303 | D | X24 | X16 | X2 | X8 | − | − | − | + | + | − | − | + | − | − | − |
| 304 | D | X24 | X15 | X2 | X8 | − | − | − | − | − | − | − | + | − | − | − |
| 305 | D | X24 | X17 | X2 | X8 | − | − | − | − | + | − | − | + | − | − | − |
| 306 | D | X24 | X19 | X2 | X3 | − | − | − | − | − | − | − | + | − | − | − |
| 307 | D | X24 | X14 | X2 | X3 | − | − | + | + | + | − | − | + | − | − | − |
| 308 | D | X24 | X20 | X2 | X3 | − | − | − | − | − | − | − | + | − | − | − |
| 309 | D | X24 | X16 | X2 | X3 | − | − | − | − | + | − | − | + | − | − | − |
| 310 | D | X24 | X15 | X2 | X3 | − | − | − | − | − | − | − | + | − | − | − |
| 311 | D | X24 | X17 | X2 | X3 | − | − | − | + | + | − | − | + | − | − | − |
| 312 | D | X24 | X14 | X2 | X1 | − | − | + | + | + | − | − | + | − | − | − |
| 313 | D | X24 | X20 | X2 | X1 | − | − | − | − | + | − | − | + | − | − | − |
| 314 | D | X24 | X16 | X2 | X1 | − | − | − | − | − | − | − | + | − | − | − |
| 315 | D | X24 | X15 | X2 | X1 | − | − | − | − | − | − | − | + | − | − | − |
| 316 | D | X24 | X19 | X2 | X2 | − | − | − | + | − | − | − | + | − | − | − |
| 317 | D | X24 | X14 | X2 | X2 | − | − | − | − | + | − | − | + | − | − | − |
| 318 | D | X24 | X20 | X2 | X2 | − | − | − | − | + | − | − | + | − | − | − |
| 319 | D | X24 | X16 | X2 | X2 | − | − | − | + | + | − | − | + | − | − | − |
| 320 | D | X24 | X15 | X2 | X2 | − | − | − | − | + | − | − | + | − | − | − |
| 321 | D | X24 | X17 | X2 | X2 | − | − | + | + | + | + | − | + | − | − | − |
| 322 | D | X24 | X22 | X5 | X8 | − | − | − | − | − | − | − | + | − | − | − |
| 323 | D | X24 | X23 | X5 | X3 | − | − | − | − | − | − | − | + | − | − | − |
| 324 | C | X24 | X21 | X5 | X8 | − | − | − | − | − | − | − | + | − | − | − |
| 325 | D | X24 | X8 | X8 | X5 | − | − | − | − | + | − | − | + | − | − | − |
| 326 | D | X24 | X23 | X8 | X5 | − | − | − | − | − | − | − | + | − | − | − |
| 327 | D | X24 | X25 | X3 | X5 | − | − | − | − | − | − | − | + | − | − | − |
| 328 | D | X24 | X8 | X3 | X5 | − | − | − | − | − | − | − | + | − | − | − |
| 329 | D | X24 | X21 | X3 | X5 | − | − | − | − | − | − | − | + | − | − | − |
| 330 | C | X24 | X8 | X8 | X5 | − | − | − | − | − | − | − | + | − | − | − |
| 331 | C | X24 | X23 | X8 | X5 | − | − | − | − | − | − | − | + | − | − | − |
| 332 | C | X24 | X25 | X3 | X5 | − | − | − | − | − | − | − | + | − | − | − |
| 333 | C | X24 | X8 | X3 | X5 | − | − | − | − | − | − | − | + | − | − | − |
| 334 | C | X24 | X23 | X3 | X5 | − | − | − | − | − | − | − | + | − | − | − |
| 335 | A | X1 | X17 | X1 | X24 | + | + | + | + | + | + | + | + | − | − | − |
| 336 | A | X1 | X17 | X1 | X24 | − | − | − | + | + | + | − | + | − | − | − |
| 337 | A | X2 | X15 | X1 | X24 | − | − | − | − | + | + | + | + | − | − | − |
| 338 | A | X2 | X15 | X2 | X24 | − | − | − | − | − | − | + | + | − | − | − |
| 339 | A | X2 | X17 | X2 | X24 | − | − | − | + | + | + | + | + | − | − | − |
| 340 | A | X2 | X15 | X3 | X24 | − | − | − | − | − | − | − | + | − | − | − |
| 341 | A | X2 | X17 | X3 | X24 | − | − | − | − | − | + | + | + | − | − | − |
| 342 | A | X3 | X15 | X1 | X24 | − | − | − | + | − | − | + | + | − | − | − |
| 343 | A | X3 | X15 | X2 | X24 | − | − | − | − | − | − | − | + | − | − | − |
| 344 | A | X3 | X15 | X2 | X24 | − | − | − | − | − | − | − | + | − | − | − |
| 345 | A | X7 | X15 | X2 | X24 | − | − | − | − | − | − | − | + | − | − | − |
| 346 | A | X7 | X15 | X2 | X24 | − | + | + | + | + | + | + | + | − | + | + |
| 347 | B | X3 | X28 | X24 | X2 | − | − | − | − | − | − | − | + | − | − | − |
| 348 | B | X3 | X29 | X24 | X2 | − | − | − | − | + | − | − | + | − | − | − |
| 349 | B | X3 | X18 | X24 | X2 | − | − | − | − | + | − | − | + | − | − | − |
| 350 | B | X3 | X14 | X24 | X2 | − | − | − | − | + | − | − | + | − | − | − |
| 351 | A | X3 | X27 | X24 | X2 | − | − | − | − | − | − | − | + | − | − | − |
| 352 | A | X3 | X28 | X24 | X2 | − | − | − | − | − | − | − | + | − | − | − |
| 353 | A | X3 | X29 | X24 | X2 | − | − | − | − | − | − | − | + | − | − | − |
| 354 | A | X3 | X18 | X24 | X2 | − | − | − | + | + | − | − | + | − | − | − |
| 355 | A | X3 | X14 | X24 | X2 | − | − | − | + | + | − | − | + | − | − | − |
| 356 | E | X20 | X24 | X9 | X30 | − | − | − | − | − | − | − | − | + | − | − |
| 357 | E | X20 | X25 | X6 | X30 | − | − | − | − | − | + | − | − | − | − | − |
| 358 | E | X20 | X25 | X9 | X30 | − | − | − | − | − | − | − | − | + | − | − |
| 359 | E | X13 | X24 | X8 | X30 | − | − | − | − | − | − | − | + | + | − | − |
| 360 | E | X19 | X25 | X8 | X30 | − | − | − | + | − | − | − | − | − | − | − |
| 361 | E | X12 | X25 | X3 | X30 | − | − | − | + | − | − | − | − | − | − | − |
| 362 | E | X12 | X25 | X9 | X30 | − | − | − | − | − | − | − | + | − | − | − |
| 363 | E | X10 | X25 | X9 | X30 | − | − | − | − | − | − | − | + | − | − | − |
| 364 | B | X8 | X20 | X3 | X2 | − | − | + | + | + | − | − | + | − | − | − |
| 365 | B | X8 | X20 | X3 | X8 | − | − | − | − | − | − | − | + | − | − | − |
| 366 | B | X8 | X20 | X2 | X2 | − | − | + | + | + | − | − | + | − | − | − |
| 367 | B | X8 | X20 | X2 | X8 | − | − | − | + | + | − | − | + | − | − | − |
| 368 | B | X8 | X20 | X2 | X3 | − | − | + | + | + | − | − | + | − | − | − |
| 369 | B | X8 | X20 | X8 | X2 | − | − | − | + | − | − | − | + | − | − | − |
| 370 | B | X8 | X20 | X8 | X8 | − | − | − | − | − | − | − | + | − | − | − |
| 371 | B | X8 | X20 | X8 | X3 | − | − | − | − | − | − | − | + | − | − | − |
| 372 | B | X8 | X15 | X3 | X2 | − | − | − | + | + | − | − | + | − | − | − |
| 373 | B | X8 | X15 | X3 | X8 | − | − | − | + | + | − | − | + | − | − | − |
| 374 | B | X8 | X15 | X3 | X3 | − | − | − | + | + | − | − | + | − | − | − |
| 375 | B | X8 | X15 | X2 | X2 | − | − | − | + | + | − | − | + | − | − | − |
| 376 | B | X8 | X15 | X2 | X8 | − | − | − | + | + | − | − | + | − | − | − |
| 377 | B | X8 | X15 | X2 | X3 | − | − | + | + | + | − | − | + | − | − | − |
| 378 | B | X8 | X15 | X8 | X2 | − | − | − | + | + | − | − | + | − | − | − |
| 379 | B | X8 | X15 | X8 | X8 | − | − | − | − | + | − | − | + | − | − | − |
| 380 | B | X8 | X15 | X8 | X3 | − | − | − | + | − | − | − | + | − | − | − |
| 381 | B | X2 | X20 | X3 | X2 | − | − | + | + | + | − | − | + | − | − | − |
| 382 | B | X2 | X20 | X3 | X8 | − | − | − | + | + | − | − | + | − | − | − |
| 383 | B | X2 | X20 | X3 | X3 | − | − | + | + | + | − | − | + | − | − | − |
| 384 | B | X2 | X20 | X2 | X2 | − | − | + | + | + | + | − | + | − | − | − |
| 385 | B | X2 | X20 | X2 | X8 | − | − | + | + | + | + | − | + | − | − | − |
| 386 | B | X2 | X20 | X2 | X3 | − | − | + | + | + | − | − | + | − | − | − |
| 387 | B | X2 | X20 | X8 | X2 | − | − | + | + | + | − | − | + | − | − | − |
| 388 | B | X2 | X20 | X8 | X8 | − | − | − | + | + | − | − | + | − | − | − |
| 389 | B | X2 | X20 | X8 | X3 | − | − | + | + | + | − | − | + | − | + | − |
| 390 | B | X2 | X15 | X3 | X2 | − | − | + | + | + | + | − | + | − | + | − |
| 391 | B | X2 | X15 | X3 | X8 | − | − | − | + | − | − | − | + | − | − | − |
| 392 | B | X2 | X15 | X3 | X3 | − | − | + | + | + | − | − | + | − | − | − |
| 393 | B | X2 | X15 | X2 | X2 | − | − | + | + | + | − | − | + | − | − | − |
| 394 | B | X2 | X15 | X2 | X8 | − | − | + | + | + | − | − | + | − | − | − |
| 395 | B | X2 | X15 | X2 | X3 | − | − | + | + | + | − | − | + | − | + | − |
| 396 | B | X2 | X15 | X8 | X2 | − | − | + | + | + | − | − | + | − | − | − |
| 397 | B | X2 | X15 | X8 | X8 | − | − | − | + | − | − | − | + | − | + | − |
| 398 | B | X2 | X15 | X8 | X3 | − | − | + | + | + | − | − | + | − | − | − |
| 399 | B | X1 | X14 | X3 | X2 | − | − | + | + | + | − | − | + | − | − | − |
| 400 | B | X1 | X14 | X3 | X8 | − | − | + | + | + | + | − | + | − | + | − |
| 401 | B | X1 | X14 | X3 | X3 | − | − | + | + | + | + | − | + | − | + | − |
| 402 | B | X1 | X14 | X2 | X2 | − | − | + | + | + | + | − | + | − | + | − |
| 403 | B | X1 | X14 | X2 | X8 | − | − | + | + | + | + | − | + | − | + | − |
| 404 | B | X1 | X14 | X2 | X3 | − | − | + | + | + | − | − | + | − | + | − |
| 405 | B | X1 | X14 | X1 | X2 | − | − | + | − | + | − | − | + | − | − | − |
| 406 | B | X1 | X14 | X1 | X8 | − | − | + | + | + | − | − | + | − | + | − |
| 407 | B | X1 | X14 | X1 | X3 | − | − | + | + | + | + | − | + | − | + | − |
| 408 | B | X1 | X16 | X3 | X2 | − | − | + | + | + | + | − | + | − | + | − |
| 409 | B | X1 | X16 | X3 | X8 | − | − | + | + | + | + | − | + | − | + | − |
| 410 | B | X1 | X16 | X3 | X3 | − | − | + | + | + | + | − | + | − | + | − |
| 411 | B | X1 | X16 | X2 | X2 | − | − | + | + | + | + | − | + | − | + | − |
| 412 | B | X1 | X16 | X2 | X8 | − | − | + | + | + | + | − | + | − | + | − |
| 413 | B | X1 | X16 | X2 | X3 | − | − | + | + | + | + | − | + | − | + | − |
| 414 | B | X1 | X16 | X1 | X2 | − | − | − | − | + | − | − | + | − | − | − |
| 415 | B | X1 | X16 | X1 | X8 | − | − | + | + | + | + | − | + | − | − | − |
| 416 | B | X1 | X16 | X1 | X3 | − | − | + | + | + | + | − | + | − | + | − |
| 417 | B | X3 | X14 | X3 | X2 | − | − | + | + | + | + | − | + | − | − | − |
| 418 | B | X3 | X14 | X3 | X8 | − | − | + | + | + | + | − | + | − | + | − |
| 419 | B | X3 | X14 | X3 | X3 | − | − | + | + | + | + | − | + | − | + | − |
| 420 | B | X3 | X14 | X2 | X2 | − | − | + | + | + | − | − | + | − | + | − |
| 421 | B | X3 | X14 | X2 | X8 | − | − | + | + | + | + | − | + | − | + | − |
| 422 | B | X3 | X14 | X2 | X3 | − | − | + | + | + | + | − | + | − | + | − |
| 423 | B | X3 | X14 | X1 | X2 | − | − | + | + | + | − | − | + | − | + | − |
| 424 | B | X3 | X14 | X1 | X8 | − | − | + | + | + | − | − | + | − | − | − |
| 425 | B | X3 | X14 | X1 | X3 | − | − | + | + | + | + | − | + | − | + | − |
| 426 | B | X3 | X16 | X3 | X2 | − | − | + | + | + | + | − | + | + | + | − |
| 427 | B | X3 | X16 | X3 | X8 | − | − | + | + | + | + | − | + | − | + | − |
| 428 | B | X3 | X16 | X3 | X3 | − | − | + | + | + | + | − | + | − | + | − |
| 429 | B | X3 | X16 | X2 | X2 | − | − | + | + | + | + | − | + | − | − | − |
| 430 | B | X3 | X16 | X2 | X8 | − | − | + | + | + | + | − | + | − | + | − |
| 431 | B | X3 | X16 | X2 | X3 | − | − | + | + | + | + | − | + | − | + | − |
| 432 | B | X3 | X16 | X1 | X2 | − | − | + | + | + | + | − | + | − | − | − |
| 433 | B | X3 | X16 | X1 | X8 | − | − | − | + | + | + | − | + | − | − | − |
| 434 | B | X3 | X16 | X1 | X3 | − | − | + | + | + | + | − | + | − | − | − |
| 435 | B | X1 | X17 | X8 | X8 | − | + | + | + | + | + | + | + | − | + | − |
| 436 | A | X2 | X20 | X3 | X2 | − | − | + | + | + | + | − | + | − | − | − |
| 437 | A | X2 | X20 | X3 | X2 | − | − | + | + | + | + | − | + | − | − | − |
| 438 | A | X2 | X20 | X3 | X8 | − | − | − | − | − | + | − | + | − | − | − |
| 439 | A | X2 | X20 | X3 | X8 | − | − | − | + | + | − | − | + | − | − | − |
| 440 | A | X2 | X20 | X3 | X3 | − | − | − | − | + | + | − | + | − | − | − |
| 441 | A | X2 | X20 | X3 | X3 | − | − | + | + | + | + | − | + | − | − | − |
| 442 | A | X2 | X20 | X2 | X2 | − | − | + | + | + | + | − | + | − | + | − |
| 443 | A | X2 | X20 | X2 | X2 | − | − | + | + | + | + | − | + | − | + | − |
| 444 | A | X2 | X20 | X2 | X8 | − | − | − | − | − | − | − | + | − | − | − |
| 445 | A | X2 | X20 | X2 | X8 | − | + | + | + | + | + | + | + | − | + | − |
| 446 | A | X2 | X20 | X2 | X3 | − | − | − | − | + | − | − | + | − | − | − |
| 447 | A | X2 | X20 | X2 | X3 | − | − | + | + | + | + | − | + | − | − | − |
| 448 | A | X2 | X20 | X8 | X2 | − | − | − | + | + | − | − | + | − | − | − |
| 449 | A | X2 | X20 | X8 | X2 | − | − | + | + | + | + | − | + | − | + | − |
| 450 | A | X2 | X20 | X8 | X8 | − | − | − | − | − | − | − | + | − | − | − |
| 451 | A | X2 | X20 | X8 | X8 | − | − | − | + | − | − | − | + | − | − | − |
| 452 | A | X2 | X20 | X8 | X3 | − | − | − | + | + | − | − | + | − | − | − |
| 453 | A | X2 | X20 | X8 | X3 | − | − | + | + | + | + | − | + | − | − | − |
| 454 | A | X2 | X15 | X3 | X2 | − | − | + | + | + | + | + | + | − | + | − |
| 455 | A | X2 | X15 | X3 | X2 | − | + | + | + | + | + | + | + | − | − | − |
| 456 | A | X2 | X15 | X3 | X8 | − | − | − | − | + | + | − | + | − | − | − |
| 457 | A | X2 | X15 | X3 | X8 | − | − | − | − | − | − | + | + | − | + | − |
| 458 | A | X2 | X15 | X3 | X3 | − | − | + | + | + | + | + | + | − | + | − |
| 459 | A | X2 | X15 | X3 | X3 | − | − | + | + | + | + | − | + | − | + | − |
| 460 | A | X2 | X15 | X2 | X2 | − | − | − | − | + | − | − | + | − | − | − |
| 461 | A | X2 | X15 | X2 | X2 | − | − | + | + | + | + | + | + | − | + | − |
| 462 | A | X2 | X15 | X2 | X8 | − | − | − | − | + | − | − | + | − | + | − |
| 463 | A | X2 | X15 | X2 | X3 | − | + | + | + | + | + | − | + | − | − | − |
| 464 | A | X2 | X15 | X2 | X3 | − | − | + | + | + | + | − | + | − | + | − |
| 465 | A | X2 | X15 | X8 | X2 | − | − | − | − | + | − | − | + | − | − | − |
| 466 | A | X2 | X15 | X8 | X2 | − | − | − | + | + | + | − | + | − | + | − |
| 467 | A | X2 | X15 | X8 | X8 | − | − | − | − | − | − | − | + | − | − | − |
| 468 | A | X2 | X15 | X8 | X8 | − | − | − | − | − | − | + | + | − | + | − |
| 469 | A | X2 | X15 | X8 | X3 | − | − | − | + | + | − | − | + | − | − | − |
| 470 | A | X2 | X15 | X8 | X3 | − | − | − | − | + | − | − | + | − | + | − |
| 471 | A | X3 | X20 | X3 | X2 | − | − | + | − | + | − | − | + | − | − | − |
| 472 | A | X3 | X20 | X3 | X2 | − | − | + | + | + | + | − | + | − | − | − |
| 473 | A | X3 | X20 | X3 | X8 | − | − | − | − | + | + | + | + | − | − | − |
| 474 | A | X3 | X20 | X3 | X8 | − | − | − | − | − | − | − | + | − | + | − |
| 475 | A | X3 | X20 | X3 | X3 | − | − | − | + | + | − | − | + | − | − | − |
| 476 | A | X3 | X20 | X3 | X3 | − | − | + | + | + | + | + | + | − | + | − |
| 477 | A | X3 | X20 | X2 | X2 | − | − | − | − | + | − | − | + | − | + | − |
| 478 | A | X3 | X20 | X2 | X2 | − | − | + | + | + | + | − | + | − | − | − |
| 479 | A | X3 | X20 | X2 | X8 | − | − | − | − | − | − | − | + | − | − | − |
| 480 | A | X3 | X20 | X2 | X8 | − | − | − | + | + | + | + | + | − | − | − |
| 481 | A | X3 | X20 | X2 | X3 | − | + | + | + | + | + | − | + | − | + | − |
| 482 | A | X3 | X20 | X2 | X3 | − | − | + | + | + | + | − | + | − | + | − |
| 483 | A | X3 | X20 | X8 | X2 | − | − | − | − | + | − | − | + | − | − | − |
| 484 | A | X3 | X20 | X8 | X2 | − | − | − | − | + | − | − | + | − | − | − |
| 485 | A | X3 | X20 | X8 | X8 | − | − | − | − | − | − | − | + | − | − | − |
| 486 | A | X3 | X20 | X8 | X8 | − | − | − | − | − | − | − | + | − | − | − |
| 487 | A | X3 | X20 | X8 | X3 | − | − | − | − | + | − | + | − | − | − | − |
| 488 | A | X3 | X20 | X8 | X3 | − | − | − | + | + | − | − | + | − | + | − |
| 489 | A | X3 | X15 | X3 | X2 | − | − | + | + | + | − | + | + | − | + | − |
| 490 | A | X3 | X15 | X3 | X2 | − | − | + | + | + | + | + | + | − | + | − |
| 491 | A | X3 | X15 | X3 | X8 | − | − | − | − | − | − | − | + | − | + | − |
| 492 | A | X3 | X15 | X3 | X8 | − | − | − | + | + | + | − | + | − | + | − |
| 493 | A | X3 | X15 | X3 | X3 | − | − | − | + | + | + | − | + | − | + | − |
| 494 | A | X3 | X15 | X3 | X3 | − | − | + | + | + | + | − | + | − | + | − |
| 495 | A | X3 | X15 | X2 | X2 | − | − | + | + | + | + | + | + | − | + | − |
| 496 | A | X3 | X15 | X2 | X2 | − | − | + | + | + | + | + | + | − | + | − |
| 497 | A | X3 | X15 | X2 | X8 | − | − | − | − | + | − | − | + | − | − | − |
| 498 | A | X3 | X15 | X2 | X8 | − | − | − | + | + | − | − | + | − | + | − |
| 499 | A | X3 | X15 | X2 | X3 | − | − | + | + | + | − | − | + | − | + | − |
| 500 | A | X3 | X15 | X2 | X3 | − | − | + | + | + | + | + | + | − | + | − |
| 501 | A | X3 | X15 | X8 | X2 | − | − | + | + | + | − | − | + | − | + | − |
| 502 | A | X3 | X15 | X8 | X2 | − | − | − | + | + | + | + | + | − | + | − |
| 503 | A | X3 | X15 | X8 | X8 | − | − | − | − | + | − | − | + | − | − | − |
| 504 | A | X3 | X15 | X8 | X8 | − | − | − | − | − | − | − | + | − | + | − |
| 505 | A | X3 | X15 | X8 | X3 | − | − | − | − | + | − | + | + | − | − | − |
| 506 | A | X3 | X15 | X8 | X3 | − | − | − | − | + | − | − | + | − | + | − |
| 507 | A | X3 | X14 | X3 | X2 | − | − | − | + | + | + | − | + | − | + | − |
| 508 | A | X3 | X14 | X3 | X2 | − | + | + | + | + | + | − | + | − | + | − |
| 509 | A | X3 | X14 | X3 | X8 | − | − | − | + | + | + | + | + | − | − | − |
| 510 | A | X3 | X14 | X3 | X8 | − | − | + | + | + | + | − | + | − | + | − |
| 511 | A | X3 | X14 | X3 | X3 | − | − | + | + | + | + | + | + | − | + | − |
| 512 | A | X3 | X14 | X2 | X2 | − | − | + | + | + | + | + | + | − | + | − |
| 513 | A | X3 | X14 | X2 | X2 | − | + | + | + | + | + | + | + | + | + | − |
| 514 | A | X3 | X14 | X2 | X8 | − | − | + | + | + | + | − | + | + | + | − |
| 515 | A | X3 | X14 | X2 | X3 | − | − | + | + | + | + | − | + | − | + | − |
| 516 | A | X3 | X14 | X2 | X3 | − | − | + | + | + | + | − | + | − | − | − |
| 517 | A | X3 | X14 | X1 | X2 | − | + | + | + | + | + | − | + | − | − | − |
| 518 | A | X3 | X14 | X1 | X8 | − | − | − | − | − | − | − | + | − | − | − |
| 519 | A | X3 | X14 | X1 | X8 | − | − | + | + | + | + | − | + | − | − | − |
| 520 | A | X3 | X14 | X1 | X3 | − | − | + | + | + | + | − | + | − | − | − |
| 521 | A | X3 | X14 | X1 | X3 | − | − | + | + | + | + | − | + | − | + | − |
| 522 | A | X3 | X16 | X3 | X2 | − | + | + | + | + | + | − | + | + | + | − |
| 523 | A | X3 | X16 | X3 | X2 | − | + | + | + | + | + | + | + | − | + | − |
| 524 | A | X3 | X16 | X3 | X8 | − | − | − | − | + | + | − | + | − | − | − |
| 525 | A | X3 | X16 | X3 | X8 | − | + | + | + | + | + | − | + | − | − | − |
| 526 | A | X3 | X16 | X3 | X3 | − | − | − | − | + | + | − | + | − | − | − |
| 527 | A | X3 | X16 | X3 | X3 | − | + | + | + | + | + | − | + | − | − | − |
| 528 | A | X3 | X16 | X2 | X2 | − | − | − | − | − | − | − | + | − | − | − |
| 529 | A | X3 | X16 | X2 | X2 | − | + | + | + | + | + | − | + | − | − | − |
| 530 | A | X3 | X16 | X2 | X8 | − | + | + | + | + | + | + | + | − | + | − |
| 531 | A | X3 | X16 | X2 | X8 | − | + | + | + | + | + | + | + | − | + | − |
| 532 | A | X3 | X16 | X2 | X3 | − | + | + | + | + | + | + | + | − | + | − |
| 533 | A | X3 | X16 | X1 | X2 | − | − | + | + | + | + | − | + | − | − | − |
| 534 | A | X3 | X16 | X1 | X2 | − | − | + | + | + | + | − | + | − | − | − |
| 535 | A | X3 | X16 | X1 | X8 | + | + | + | + | + | + | + | + | − | − | − |
| 536 | A | X3 | X16 | X1 | X8 | − | + | + | + | + | + | − | + | − | − | − |
| 537 | A | X3 | X16 | X1 | X3 | − | − | + | + | + | + | − | + | − | + | − |
| 538 | A | X3 | X16 | X1 | X3 | − | + | + | + | + | + | − | + | + | + | + |
| 539 | F | — | X14 | X3 | X3 | − | − | − | + | − | − | − | + | − | − | − |
| 540 | F | — | X14 | X3 | X2 | − | − | − | + | − | − | − | + | − | − | − |
| 541 | F | — | X17 | X3 | X2 | − | − | − | − | − | − | − | + | − | − | − |
| 542 | F | — | X17 | X3 | X3 | − | − | − | − | − | − | − | + | − | − | − |
| 543 | E | X8 | X15 | X2 | X24 | − | − | − | − | − | − | − | + | − | − | − |
| 544 | F | — | X20 | X3 | X1 | − | + | + | − | − | − | − | + | − | − | − |
| 545 | B | X2 | X20 | X2 | X24 | − | − | − | − | − | − | − | + | − | − | − |
| 546 | B | X2 | X20 | X2 | X24 | − | − | − | − | + | − | − | − | − | − | − |
| 547 | B | X2 | X16 | X2 | X24 | − | − | − | − | − | + | − | + | − | − | − |
| 548 | B | X7 | X14 | X1 | X24 | − | − | − | − | + | − | − | + | − | − | − |
| 549 | B | X7 | X14 | X1 | X24 | − | − | − | − | + | − | − | − | − | − | − |

Key to Blocks Table 3: Results.

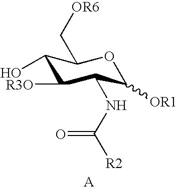

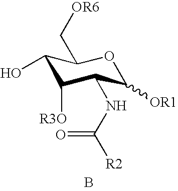

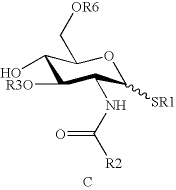

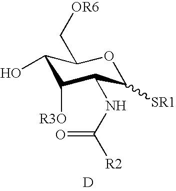

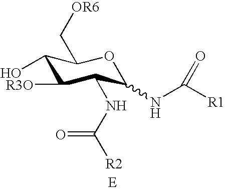

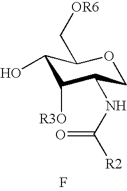

Key to Table 3: Results

“+” indicates greater than 50% inhibition at 10 μM, “−” indicates less than 50% inhibition at 10 μM. “P” indicates precipitation

X1-X30 are sidearms selected from the figure below.

[image]

Throughout the specification and the claims (if present), unless the context requires otherwise, the term “comprise”, or variations such as “comprises” or “comprising”, will be understood to apply the inclusion of the stated integer or group of integers but not the exclusion of any other integer or group of integers.

Throughout the specification and claims (if present), unless the context requires otherwise, the term “substantially” or “about” will be understood to not be limited to the value for the range qualified by the terms.

It should be appreciated that various other changes and modifications can be made to any embodiment described without departing from the spirit and scope of the invention.

1. A method of identifying biologically active compounds with defined selectivity profile comprising:
(a) designing a library of compounds of formula 1 to scan molecular diversity; and
(b) assaying the library of compounds in at least two different biological targets; wherein formula 1 represents: 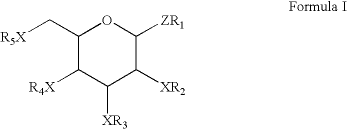
wherein the ring may be of any configuration;
Z is, oxygen, CH₂, C(O), C(O)NR^{A}, NH, NR^{A} or hydrogen, in the case where Z is hydrogen then R₁ is not present, R^{A} is selected from the set defined for R₁ to R₅, or wherein Z and R₁ together form a heterocycle; X is oxygen or nitrogen;
When X is oxygen, R₁ to R₅ are independently selected from the group which includes but is not limited to H or an C1 to C20 alkyl or acyl; C2 to C20 alkenyl, alkynyl, heteroalkyl; C5 to C20 aryl, heteroaryl, arylalkyl or heteroarylalkyl, which is optionally substituted, and can be branched or linear wherein R₁ to R₅ optional substituents are selected from the group consisting of OH, NO, NO₂, NH₂, N₃, halogen, CF₃, CHF₂, CH₂F, nitrile, alkoxy, aryloxy, amidine, guanidiniums, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoaryl, aminoheteroaryl, thioalkyl, thioaryl or thioheteroaryl, which may be further substituted; or
when X is nitrogen, each X may combine independently with the corresponding R₂ to R₅ to form an azide, or wherein each X may also combine independently with any one of corresponding R₂ to R₅ to form a heterocycle, wherein R₁ to R₅ are independently selected from the group which includes but is not limited to H or an C1 to C20 alkyl or acyl; C2 to C20 alkenyl, alkynyl, heteroalkyl; C5 to C20 aryl, heteroaryl, arylalkyl or heteroarylalkyl, which is optionally substituted, and can be branched or linear wherein R₁-R₅ optional substituents are selected from the group consisting of OH, NO, NO₂, NH₂, N₃, halogen, CF₃, CHF₂, CH₂F, nitrile, alkoxy, aryloxy, amidine, guanidiniums, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoaryl, aminoheteroaryl, thioalkyl, thioaryl or thioheteroaryl, which may be further substituted.

2. The method according to claim 1 wherein at least one X is nitrogen.

3. The method according to claim 1 wherein two of X is nitrogen.

4. The method according to claim 1 wherein X and R₂ combine to form a heterocycle.

5. The method of claim 1 wherein R₁ to R₅ optional substituents are selected from the group consisting of OH, NO, NO₂, NH₂, N₃, halogen, CF₃, CHF₂, CH₂F, nitrile, alkoxy, aryloxy, amidine, guanidiniums, aryl, cycloalkyl, heteroalkyl, heteroaryl, aminoalkyl, aminodialkyl, aminotrialkyl, aminoacyl, substituted or unsubstituted imine, sulfate, sulfonamide, phosphate, phosphoramide, hydrazide, hydroxamate, hydroxamic acid, heteroaryloxy, aminoaryl, aminoheteroaryl, thioalkyl, thioaryl or thioheteroaryl, which may be further substituted.

6. The method according to claim 1, wherein the library of compounds is selected from compounds of formula II, 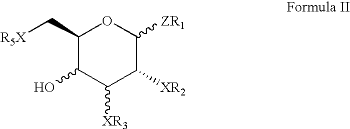
wherein Z is sulphur, oxygen, CH₂, C(O), C(O)NR^{A}, NH, NR^{A} or hydrogen, in the case where Z is hydrogen then R₁ is not present, R^{A} is selected from the set defined for R₁ to R₅, or wherein Z and R₁ together form a heterocycle;
X is oxygen or nitrogen, when X is nitrogen, each X may combine independently with the corresponding R₂ to R₅ to form an azide, or wherein each X may also combine independently with any one of corresponding R₂ to R₅ to form a heterocycle;
R₁ to R₅ are independently selected from the group which includes H or an C1 to C20 alkyl or acyl; C2 to C20 alkenyl, alkynyl, heteroalkyl; C5 to C20 aryl, heteroaryl, arylalkyl or heteroarylalkyl, which may be substituted, and can be branched or linear.

7. The method according to claim 1, wherein the library of compounds is selected from compounds of formula III, 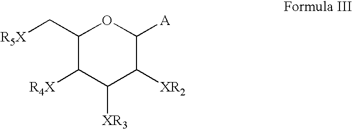
wherein A is defined as hydrogen, or OR₁,
R₁ to R₅ are independently selected from the group which includes H or an C1 to C20 alkyl or acyl; C2 to C20 alkenyl, alkynyl, heteroalkyl; C5 to C20 aryl, heteroaryl, arylalkyl or heteroarylalkyl, which may be substituted, and can be branched or linear,
X is oxygen or nitrogen, when X is nitrogen, each X may combine independently with the corresponding R₂ to R₅ to form an azide, or wherein each X may also combine independently with any one of corresponding R₂ to R₅ to form a heterocycle.

8. The method according to claim 1, wherein the library of compounds is selected from compounds of formula IV, 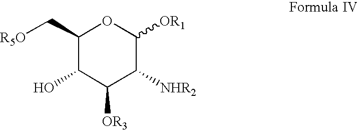
wherein R₁, R₂, R₃ and R₅ are independently selected from the group which includes H or an C1 to C20 alkyl or acyl; C2 to C20 alkenyl, alkynyl, heteroalkyl; C5 to C20 aryl, heteroaryl, arylalkyl or heteroarylalkyl, which may be substituted, and can be branched or linear.

9. The method according to claim 1, wherein the library of compounds is selected from compounds of formula V, 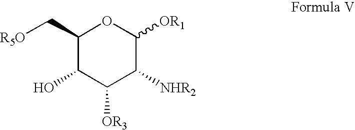
wherein R₁, R₂, R₃ and R₅ are independently selected from the group which includes H or an C1 to C20 alkyl or acyl; C2 to C20 alkenyl, alkynyl, heteroalkyl; C5 to C20 aryl, heteroaryl, arylalkyl or heteroarylalkyl, which may be substituted, and can be branched or linear.

10-11. (canceled)

12. A method according to claim 1 wherein the library of compounds is selected from compounds of formula VIII, 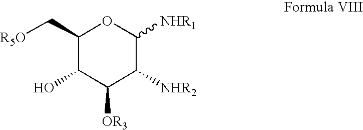
wherein R₁, R₂, R₃ and R₅ are independently selected from the group which includes H or an C1 to C20 alkyl or acyl; C2 to C20 alkenyl, alkynyl, heteroalkyl; C5 to C20 aryl, heteroaryl, arylalkyl or heteroarylalkyl, which may be substituted, and can be branched or linear.

13. The method according to claim 1, wherein the library of compounds is selected from compounds of formula IX, 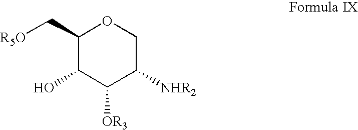
wherein R₂, R₃ and R₅ are independently selected from the group which includes H or an C1 to C20 alkyl or acyl; C2 to C20 alkenyl, alkynyl, heteroalkyl; C5 to C20 aryl, heteroaryl, arylalkyl or heteroarylalkyl, which may be substituted, and can be branched or linear.

14. The method according to claim 1 wherein the biological assays involve Peptide Ligand class of GPCRs.

15. The method according to claim 14 wherein biological assays involve opioid, melanocortin, melanin-concentrating hormone, neurokinin, neuropeptide and urotensin receptors.

16. The method according to claim 15 wherein biological assays involve δ-opioid (DOP), κ-Opioid (KOP), Melanocortin MC3, Melanocortin MC4, Melanocortin MC5, Melanin-Concentrating Hormone (MCH1), μ-opioid (MOP), Neurokinin (NK1), Neuropeptide Y (NPY-Y1), Opioid (ORL1) and urotensin (UR2) receptors.

17. A library of compounds selected from compounds of formula 1, wherein formula 1 represents: 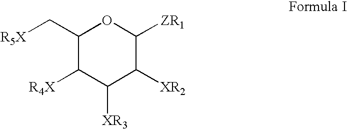
wherein the ring may be of any configuration,
Z is oxygen, CH₂, C(O), C(O)NR^{A}, NH, NR^{A} or hydrogen, in the case where Z is hydrogen then R₁ is not present, R^{A} is selected from the set defined for R₁ to R₅, or wherein Z and R₁ together form a heterocycle; X is oxygen or nitrogen;
when X is oxygen R₁ to R₅ are independently selected from the group which includes but is not limited to H or an C1 to C20 alkyl or acyl; C2 to C20 alkenyl, alkynyl heteroalkyl; C5 to C20 aryl, heteroaryl, arylalkyl or heteroarylalkyl, which is optionally substituted, and can be branched or linear wherein R₁ to R₅ optional substituents are selected from the group consisting of OH, NO, NO₂, NH₂, N₃, halogen, CF₃, CHF₂, CH₂F, nitrile, alkoxy, aryloxy, amidine, guanidiniums, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoaryl, aminoheteroaryl, thioalkyl, thioaryl or thioheteroaryl, which may be further substituted: or
when X is nitrogen, each X may combine independently with the corresponding R₂ to R₅ to form an azide, or wherein each X may also combine independently with any one of corresponding R₂-R₅ to form a heterocycle, wherein R₁ to R₅ are independently selected from the group which includes but is not limited to H or an C1 to C20 alkyl or acyl; C2 to C20 alkenyl, alkynyl, heteroalkyl; C5 to C20 aryl, heteroaryl, arylalkyl or heteroarylalkyl, which is optionally substituted, and can be branched or linear wherein R₁ to R₅ optional substituents are selected from the group consisting of OH, NO, NO₂, NH₂, N₃, halogen, CF₃, CHF₂, CH₂F, nitrile, alkoxy, aryloxy, amidine, guanidiniums, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoaryl, aminoheteroaryl, thioalkyl, thioaryl or thioheteroaryl, which may be further substituted; and
wherein said library is designed to scan for molecular diversity.

18. The library of compounds according to claim 17, wherein the compounds are selected from compounds of formula II, wherein formula II represents: 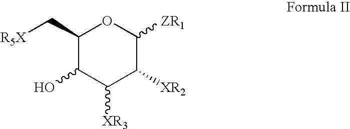
wherein Z is oxygen, CH₂, C(O), C(O)NR^{A}, NH, NR^{A} or hydrogen, in the case where Z is hydrogen then R₁ is not present R^{A} is selected from the set defined for R₁ to R₅or wherein Z and R₁ together form a heterocycle;
X is oxygen or nitrogen, when X is nitrogen, each X may combine independently with the corresponding R₂ to R₅ to form an azide, or wherein each X may also combine independently with any one of corresponding R₂ to R₅ to form a heterocycle;
R₁ to R₅ are independently selected from the group which includes H or an C1 to C20 alkyl or acyl; C2 to C20 alkenyl, alkynyl heteroalkyl: C5 to C20 aryl heteroaryl, arylalkyl or heteroarylalkyl, which may be substituted, and can be branched or linear.

19. The library of compounds according to claim 17, wherein the compounds are selected from compounds of formula III, wherein formula III represents: 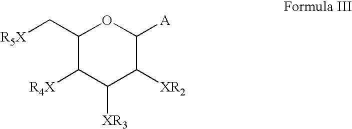
wherein A is defined as hydrogen SR₁, or OR₁,
R₁ to R₅ are independently selected from the group which includes H or an C1 to C20 alkyl or acyl: C2 to C20 alkenyl, alkynyl heteroalkyl: C5 to C20 aryl heteroaryl, arylalkyl or heteroarylalkyl, which may be substituted, and can be branched or linear, X is oxygen or nitrogen, when X is nitrogen, each X may combine independently with the corresponding R₂ to R₅ to form an azide, or wherein each X may also combine independently with any one of corresponding R₂ to R₅ to form a heterocycle.

20. The library of compounds according to claim 17, wherein the compounds are selected from compounds of formula IV, wherein formula IV represents: 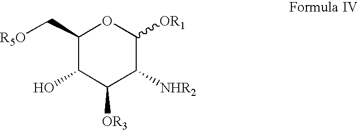
wherein R₁, R₂, R₃ and R₅ are independently selected from the group which includes H or an C1 to C20 alkyl or acyl: C2 to C20 alkenyl, alkynyl, heteroalkyl: C5 to C20 aryl, heteroaryl, arylalkyl or heteroarylalkyl, which may be substituted, and can be branched or linear.

21. The library of compounds according to claim 17, wherein the compounds are selected from compounds of formula V, wherein formula V represents: 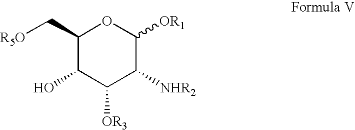
wherein R₁, R₂, R₃ and R₅ are independently selected from the group which includes H or an C1 to C20 alkyl or acyl: C2 to C20 alkenyl, alkynyl, heteroalkyl; C5 to C20 aryl, heteroaryl, arylalkyl or heteroarylalkyl, which may be substituted, and can be branched or linear.

22-23. (canceled)

24. The library of compounds according to claim 17, wherein the compounds are selected from compounds of formula VIII, wherein formula VIII represents: 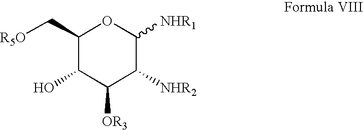
wherein R₁, R₂, R₃ and R₅ are independently selected from the group which includes H or an C1 to C20 alkyl or acyl: C2 to C20 alkenyl, alkynyl, heteroalkyl: C5 to C20 aryl, heteroaryl, arylalkyl or heteroarylalkyl, which may be substituted, and can be branched or linear.

25. The library of compounds according to claim 17, wherein the compounds are selected from compounds of formula IX, wherein formula IX represents: 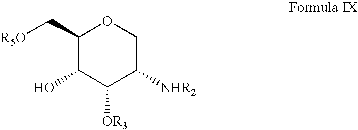
wherein R₂, R₃ and R₅ are independently selected from the group which includes H or an C1 to C20 alkyl or acyl: C2 to C20 alkenyl, alkynyl, heteroalkyl: C5 to C20 aryl, heteroaryl, arylalkyl or heteroarylalkyl, which may be substituted, and can be branched or linear.

26. A biologically active compound identified by the method of claim 1.

27. A compound according to formula I in which at least one X is nitrogen, and the at least one X is combined with the corresponding R₁ to R₅ to form a heterocycle,
wherein formula I represents: 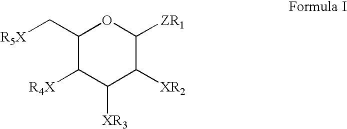
wherein the ring may be of any configuration;
Z is oxygen, CH₂, C(O), C(O)NR^{A}, NH, NR^{A} or hydrogen, in the case where Z is hydrogen then R₁ is not present, R^{A} is selected from the set defined for R₁ to R₅, or wherein Z and R₁ together form a heterocycle; X is oxygen or nitrogen;
when X is oxygen, R₁ to R₅ are independently selected from the group which includes but is not limited to H or an C1 to C20 alkyl or acyl, C2 to C20 alkenyl, alkynyl, heteroalkyl; C5 to C20 aryl, heteroaryl, arylalkyl or heteroarylalkyl, which is optionally substituted, and can be branched or linear wherein R₁-R₅ optional substituents are selected from the group consisting of OH, NO, NO₂, NH₂, N₃, halogen, CF₃, CHF₂, CH₂F, nitrile, alkoxy, aryloxy, amidine, guanidiniums, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoaryl, aminoheteroaryl, thioalkyl, thioaryl or thioheteroaryl, which may be further substituted; or
when X is nitrogen, each X may combine independently with the corresponding R₂ to R₅ to form an azide, or wherein each X may also combine independently with any one of corresponding R₂ to R₅ to form a heterocycle, wherein R₁ to R₅ are independently selected from the group which includes but is not limited to H or an C1 to C20 alkyl or acyl; C2 to C20 alkenyl, alkynyl, heteroalkyl; C5 to C20 aryl, heteroaryl, arylalkyl or heteroarylalkyl, which is optionally substituted, and can be branched or linear wherein R₁ to R₅ optional substituents are selected from the group consisting of OH, NO, NO₂, NH₂, N₃, halogen, CF₃, CHF₂, CH₂F, nitrile, alkoxy, aryloxy, amidine, guanidiniums, aryl, cycloalkyl, heteroalkyl, heteroaryl, aminoalkyl, aminodialkyl, aminotrialkyl, aminoacyl, carbonyl, substituted or unsubstituted imine, sulfate, sulfonamide, phosphate, phosphoramide, hydrazide, hydroxamate, hydroxamic acid, heteroaryloxy, aminoaryl, aminoheteroaryl, thioalkyl, thioaryl or thioheteroaryl, which may be further substituted.

28. A compound according to claim 27 wherein X and R₂ combine to form a heterocycle.

29. A compound according to claim 28, wherein the heterocycle is heteroaryl.

30. A compound according to claim 29, wherein the heteroaryl is selected from triazoles, benzimidazoles, benzimidazolone, benzimidazolothione, imidazole, hydantoine, thiohydantoine and purine.
